SEQUENCE LISTING (44 sequences)

SEQ ID NO: 1 - DNA, 11 bp, unknown
  synthetic
ggtctcnnnnn

SEQ ID NO: 2 - DNA, 11 bp, unknown
  synthetic
cgtctcnnnnn

SEQ ID NO: 3 - DNA, 10 bp, unknown
  synthetic
gtctcnnnnn

SEQ ID NO: 4 - DNA, 24 bp, unknown
  primer
cgatctgtctatttcgttcatcca

SEQ ID NO: 5 - DNA, 24 bp, unknown
  primer
ggcaacaattaatagactggatgg

SEQ ID NO: 6 - DNA, 24 bp, unknown
  primer
tttgaaattgattcactagaacat

SEQ ID NO: 7 - DNA, 24 bp, unknown
  primer
aaggaaacttgtttaaatgataac

SEQ ID NO: 8 - DNA, 24 bp, unknown
  primer
agagatacaaaatacactgaagag

SEQ ID NO: 9 - DNA, 39 bp, unknown
  primer
tcatatacaacagatagaggagcatttgaatactgggtt

SEQ ID NO: 10 - DNA, 39 bp, unknown
  primer
aacccagtattcaaatgctcctctatctgttgtatatga

SEQ ID NO: 11 - DNA, 39 bp, unknown
  primer
tcatatacaacagatagagacgcatttgaatactgggtt

SEQ ID NO: 12 - DNA, 39 bp, unknown
  primer
aacccagtattcaaatgcgtctctatctgttgtatatga

SEQ ID NO: 13 - DNA, 42 bp, unknown
  primer
agctatattgaaaaagaagacagaaatgccttattagtaata

SEQ ID NO: 14 - DNA, 42 bp, unknown
  primer
tattactaataaggcatttctgtcttctttttcaatatagct

SEQ ID NO: 15 - DNA, 42 bp, unknown
  primer
agctatattgaaaaagaaaacggaaatgccttattagtaata

SEQ ID NO: 16 - DNA, 42 bp, unknown
  primer
tattactaataaggcatttccgttttctttttcaatatagct

SEQ ID NO: 17 - DNA, 39 bp, unknown
  primer
ctatataatcatgatagagatgcttttatgtggtggtca

SEQ ID NO: 18 - DNA, 39 bp, unknown
  primer
tgaccaccacataaaagcatctctatcatgattatatag

SEQ ID NO: 19 - DNA, 45 bp, unknown
  primer
aattctaaggatataaatgagggaaagttaattaaatttgacact

SEQ ID NO: 20 - DNA, 45 bp, unknown
  primer
agtgtcaaatttaattaactttccctcatttatatccttagaatt

SEQ ID NO: 21 - DNA, 45 bp, unknown
  primer
aattctaaggatataaatgaggataagttaattaaatttgacact

SEQ ID NO: 22 - DNA, 45 bp, unknown
  primer
agtgtcaaatttaattaacttatcctcatttatatccttagaatt

SEQ ID NO: 23 - DNA, 39 bp, unknown
  primer
tcgtatacaaaagatagagatgcatatgaatattggagc

SEQ ID NO: 24 - DNA, 39 bp, unknown
  primer
gctccaatattcatatgcatctctatcttttgtatacga

SEQ ID NO: 25 - DNA, 42 bp, unknown
  primer
gattataatgaaaaagaagatggaaatataaaagcaaacctc

SEQ ID NO: 26 - DNA, 42 bp, unknown
  primer
gaggtttgcttttatatttccatcttctttttcattataatc

SEQ ID NO: 27 - DNA, 23 bp, unknown
  synthetic
ggtctcgcggtatcattgcagca

SEQ ID NO: 28 - DNA, 20 bp, unknown
  primer
tgctgcaatgataccgannn

SEQ ID NO: 29 - DNA, 21 bp, unknown
  synthetic
cgtgggtctcgcggtatcatt

SEQ ID NO: 30 - DNA, 19 bp, unknown
  primer
cgtgggtctcgcggtannn

SEQ ID NO: 31 - protein, 524 aa, unknown
  Bacillus stearothermophilus 6-55
AEYGQGHPIFLEYAEQIIQHKEYQGMPDLRYPDGRIQWEAPSNRKSGIFKDTNIKRRKWWEQKAISIGIDPSSNQWISKTAKLIHPTMRKPCKKCGRIMDLRYSYPTKNLIKRIRKLPYVDESFEIDSLEHILKLIKRLVLQYGDKVYDDLPKLLTCKAVKNIPRLGNDLDTWLNWIDSVYIPSEPSMLSPGAMANPPDRLDGFHSLNECCRSHADRGRWEKNLRSYTTDRRAFEYWVDGDWVAADKLMGLIRTNEQIKKETCLNDNHPGPCSADHIGPISLGFVHRPEFQLLCNSCNSAKNNRMTFSDVQHLINAENNGEEVASWYCKHIWDLRKHDVKNNENALRLSKILRDNRHTAMFILSELLKDNHYLFLSTFLGLQYAERSVSFSNIKIENHIITGQISEQPRDTKYTEEQKARRMRIGFEALKSYIEKENRNALLVINDKIIDKINEIKNILQDIPDEYKLLNEKISEQFNSEEVSDELLRDLVTHLPTKESEPANFKLARKYLQEIMEIVGDELSK

SEQ ID NO: 32 - protein, 525 aa, unknown
  Bacillus stearothermophilus NUB36
AKYGRGKFLPHQNYIDYMHFIVNHKNYSGMPNAIGEDGRINWQVSSGKTTSFYEYYQARFEWWEKKADELNLPGTGNSNKRFSLAARLIHPTGQRPCRLCGKYQYVGYMYVSHNLYKRWSKITGREDLFFKKQNIIEAANIFKSIMGEQALINELTTIFPERKDYFNRLPNIEDFFVSSSHIKNNGNYISPGFMANPPDRLDGFHDYGICCRKEKDPGRHDDNMRLYNHDRRAFMWWSEGDWALADALYNKAGAGKCADPDCQKEVEKISPDHVGPISCGFKQIPFFKPLCASCNSAKNRRFSYQDVKELLKYENYTGDSVASWQVRALWDNCKHLVKNDDDSKLLSNLMRSLQDYYLRSLYKLYSNGFAHLLSYFLTPEYAHYKITFEGLNTSTLEYERYYKTFKKTKSTSSLAARIVRIAFEELEIYNSKDINERKLIKFDTSSWEKDFENIISYATKNLSLDEEASKWNKVLTDKNLSSTEKDKKISSLLEDKNYEVNKKQFYILKDLLVEHFNKIGEQIAK

SEQ ID NO: 33 - DNA, 33 bp, unknown
  synthetic
aagctgtgaccgtctccgggagctgcatgtgtc

SEQ ID NO: 34 - DNA, 16 bp, unknown
  synthetic
gggagctgcatgtgtc

SEQ ID NO: 35 - DNA, 33 bp, unknown; identical to SEQ ID NO: 33
  synthetic

SEQ ID NO: 36 - DNA, 25 bp, unknown
  primer
aagctgtgaccgtctccgggaannn

SEQ ID NO: 37 - protein, 470 aa, unknown
  Bacillus stearothermophilus 6-55
EYGQGHPIFLEYAEQIIQHKEYQGMPDLRYPDGRIQWEAPSNRKSGIFKDTNIKRRKWWEQKAISIGIDPSSNQWISKTAKLIHPTMRKPCKKCGRIMDLRYSYPTKNLIKRIRKLPYVDESFEIDSLEHILKLIKRLVLQYGDKVYDDLPKLLTCKAVKNIPRLGNDLDTWLNWIDSVYIPSEPSMLSPGAMANPPDRLDGFHSLNECCRSHADRGRWEKNLRSYTTDRRAFEYWVDGDWVAADKLMGLIRTNEQIKKETCLNDNHPGPCSADHIGPISLGFVHRPEFQLLCNSCNSAKNNRMTFSDVQHLINAENNGEEVASWYCKHIWDLRKHDVKNNENALRLSKILRDNRHTAMFILSELLKDNHYLFLSTFLGLQYAERSVSFSNIKIENHIITGQISEQPRDTKYTEEQKARRMRIGFEALKSYIEKENRNALLVINDKIIDKINEIKNILQDIPDEYKLLNE

SEQ ID NO: 38 - protein, 449 aa, unknown
  Bacillus stearothermophilus A664
EEREWHPKFIEYMDFIIQHPNYKGLPITKKSDGSWSWFGTKKTQIGKARIAWCENKAKELGFPIEPGVYANVMREIHPTKWKVCQTCGHSMSIYYHYPSANFLKALKKEFGVEYTEVDHIADIWDDLLSRGFSNNKIASFLIKKGELDLNAKTSSKDEVIYELESVCRNKGKKILSPGAMSNFPDRFDGFHTYNRCCRASQDKGRSKENLKSYTKDRRAYEYWSDGNIHAANQFMGSPFFNNISADHIGPISLGFVHDPRYLQPMSGGDNSSKRDRLQLDDIEKIIETEKRTNVYPMSWYSKLIWEYIKKNYSTHKSLISGVYRDALKQNMSNFMYILWYILEHCNQDGEHFLEEALLKPNYDYFQYSYTFNELGEIVSINPRHFTDRNQYETERYKRIAFESVYDYNEKENRNIKANLIDNEQRMLNKLCQEISSGVPVEQCKKLLIE

SEQ ID NO: 39 - DNA, 23 bp, unknown
  synthetic
ggtctcgcggtatcattgcagca

SEQ ID NO: 40 - DNA, 20 bp, unknown
  primer
tgctgcaatgataccgannn

SEQ ID NO: 41 - DNA, 21 bp, unknown
  synthetic
cgtgggtctcgcggtatcatt

SEQ ID NO: 42 - DNA, 19 bp, unknown
  primer
cgtgggtctcgcggtannn

SEQ ID NO: 43 - DNA, 11 bp, unknown
  synthetic
cgannnnntgc

SEQ ID NO: 44 - DNA, 11 bp, unknown
  synthetic
acnnnngtayc

(19) United States
(12) Patent Application Publication
(10) Pub. No.: US 2005/0136462 A1
(43) Pub. Date: Jun. 23, 2005

(54) Method for engineering nicking enzymes

(75) Inventors: Zhenyu Zhu, Beverly, MA (US); Shuang-yong Xu, Lexington, MA (US)

Correspondence Address: NEW ENGLAND BIOLABS, INC., 32 TOZER ROAD, BEVERLY, MA, 01915, US

(73) Assignee: New England Biolabs, Inc., Beverly, MA (US)

(21) Appl. No.: 11/013,235

(22) Filed: Dec. 15, 2004

Related U.S. Application Data
(60) Provisional application No. 60/531,064, filed on Dec. 19, 2003.

(51) Int. Cl. C12Q 1/68; C12N 9/22; C12N 15/85

(52) U.S. Cl. 435/6; 435/455; 435/199

(57) ABSTRACT

Methods are provided for identifying novel strand-specific nicking endonucleases by means of in vitro backcrosses of mutagenized restriction endonuclease genes with their wild-type counterpart and identifying the resulting nicking endonucleases by their cleavage activity and their strand specificity. Examples of nicking endonucleases identified by this method include Nt.Bsa I and Nb.BsaI, Nt.BsmAI and Nb.BsmAI and Nt.BsmBI.

### CROSS REFERENCE

This application gains priority from provisional application U.S. Ser. No. 60/531,064 filed Dec. 19, 2003 herein incorporated by reference.

### BACKGROUND OF THE INVENTION

There are over 240 Type II restriction endonucleases with unique specificities discovered so far from bacterial and viral sources that are available for research and diagnostic applications. However, only seven nicking enzymes are commercially available, N.BstNBI, N.AlwI, N.BbvCIA, and N.BbvCIB (NEB catalog, 2002/2003) (New England Biolabs, Inc.); N.Bpu10I (Fermentas catalog, 2002, Fermentas, Inc., Hanover, Md.); and N.CviQXI (CviNY2A) and N.CviPII (CviNYSI) Megabase Research Products, Lincoln, Nebr. (www.cvienzymes.com). Additionally, there are a number of phage encoded nicking enzymes such as the gene II protein (gpII) of bacteriophage f1 that are essential for viral DNA replications. Gene II protein introduces a nick in the (+) strand to initiate rolling circle replication. It is also involved in ligating the displaced (+) strand to generate single-strand phage DNA (Geider et al., J. Biol. Chem. 257:6488-6493 (1982); Higashitani et al., J. Mol. Biol. 237:388-400 (1994)). The gpII protein and exonuclease III can be combined and used to generate single-stranded DNA for mutagenesis and as a template for new DNA strand synthesis in vitro.

Naturally occurring nicking enzymes, N.CviQXI (CviNY2A) and N.CviPII (CviNYSI) have been obtained from the lysates of Chlorella algae viruses, (Zhang, Y. et al., Virology 240:366-375 (1998); Xia, Y. et al., Nucl. Acids Res. 16:9477-9487 (1988)) and nicking endonucleases, N.BstNBI and N.BstSEI have been obtained from bacteria (Morgan R. D. et al., Biol. Chem. 381:1123-5 (2000); Abdurashitov et al., Mol. Biol. (Mosk) 30:1261-1267 (1996)). A limited number of nicking enzymes have also been obtained by altering Type IIA restriction enzymes (U.S. Pat. No. 6,191,267; U.S. Pat. No. 6,395,523; EP1176204).

Nicking endonucleases (enzymes) described above have many applications in genetic engineering (see for example, U.S. Pat. No. 6,660,475 and WO 03/087301). Consequently, it would be useful to have access to an expanded supply of nicking endonucleases of varying specificity. Techniques have been described for modifying restriction endonucleases to give rise to nicking endonucleases. However, these techniques have limitations that would be desirable to circumvent.

For example, Xu Y. et al. (Proc. Natl. Acad. Sci. USA, 98:12990-12995 (2001)) describe a method for engineering a nicking endonuclease N.AlwI by domain swapping. This approach has the disadvantage of requiring prior knowledge of the location of the dimerization domain between AlwI and a homologous nicking enzyme, N.BstNBI. The newly engineered enzyme N.AlwI cannot form a dimer and only nicks DNA as a monomer, a property conferred by the domain originating in N.BstNBI.

Besnier, C. E. and Kong, H. EMBO Report 2:782-786 (2001), Kong, H. et al. U.S. Pat. No. 6,395,523, (2002) used site-directed mutagenesis to obtain MlyI variants. The dimerization function of the wild-type enzyme was disrupted.

Stahl F. et al. (Proc. Natl. Acad. Sci. USA, 93:6175-80 (1996)) disclosed a method for making a nicking endonuclease that was non-specific for a particular DNA strand in a duplex. This method involved creating EcoRV mutants, which were modified by fusion with different peptide tags. The EcoRV nicking enzyme was then constructed from a subunit with an inactive catalytic activity with a second subunit having a deficiency in DNA binding. Because the mutants produced by this method were non-specific with respect to which strand to nick their usefulness for DNA manipulation was adversely affected.

Other mutagenesis studies on EcoRI restriction endonuclease also produced some mutants that preferentially nicked DNA rather than cleave the duplex DNA. For example, EcoRI R200C mutant protein generated a higher proportion of nicked-circular DNA than does the wt EcoRI enzyme (Heitman, J. and Medel, P., Proteins: Structure, Functions, and Genetics 7:185-197 (1990)). Again, due to the non-specific nature of strand nicking, such mutants are not widely used in molecular biology applications.

Janulaitis A. et al. (European patent EP 1176204 A1) described a method for engineering sequence specific DNA nicking enzymes from Type IIT restriction endonucleases. Type IIT includes enzymes that are composed of heterodimeric subunits such as Bpu10I, BbvCI, and BslI. By inactivation of the catalytic activity of the a subunit of a Type IIT restriction endonuclease and formation of heterodimer with the wild-type β subunit, a sequence-specific nicking enzyme (nickase) could be constructed.

Heiter D. et al. (U.S. patent application 2003-0100094) described a method for engineering BbvCI and other restriction endonucleases into nicking enzymes suitable for Type II restriction endonucleases with clearly defined and conserved catalytic sites.

Heitman and Model describe a method for random mutagenesis of EcoRI restriction endonuclease (Heitman, J. and Model, P., EMBO J. 9:3369-3378 (1990)). The method utilized mutagen nitrosoguanidine to treat E. coli cells carrying the ecoRIR gene. The plasmid carrying the ecoRIR gene was also mutagenized by passing the plasmid through a mutD mutator strain. EcoRI variants (null mutants, low activity mutants, relaxed-specificity mutants, and nicking mutants) were isolated.

Xu and Schildkraut describe a random mutagenesis method to mutagenize the bamHIR gene with hydroxylamine (Xu, S.-y. and Schildkraut, I., J. Biol. Chem. 266:4425-4429 (1991)). Following the random mutagenesis, BamHI variants (null mutants, low activity mutants, catalytic mutants with DNA binding activity and deficient in cleavage activity) were isolated.

Different sub-types of Type II restriction endonucleases have been classified by Roberts R. J. et al. (Nucl. Acids Res. 31:1805-1812 (2003)). Type IIA restriction endonucleases have asymmetric DNA recognition sequences. The cleavage position can be within the recognition sequence or outside (downstream) of recognition sequences. Examples are BsmI (GAATGC 1/−1, top strand cleavage one base downstream of the recognition sequence, bottom strand cleavage within the recognition sequence), AciI (CCGC −3/−1), BssSI (CACGAG −5/−1), and BsaI (GGTCTC 1/5). Type IIA includes Type IIG, Type IIH, Type IIS, and Type IIT restriction endonucleases with asymmetric DNA recognition sequence. Many enzymes can fall into more than one sub-type. For example, BsaI is both a Type IIA and Type IIS enzyme.

Type IIS restriction endonucleases have asymmetric DNA recognition sequences and cleave DNA outside of their recognition sequences, i.e. cleavage 1 to 20 bases outside of DNA recognition sequence. Examples are BsmAI (GTCTC 1/5), BsmBI (CGTCTC 1/5), FokI (GGATG 9/13), and SapI (GCTCTTC 1/4).

Type IIG restriction endonucleases contain a fusion of endonuclease domain and methylase domain. Therefore, Type IIG enzymes have both endonuclease and methylase activities and the activities may be stimulated by addition of AdoMet. The DNA recognition sequences of Type IIG enzymes could be symmetric or asymmetric. Examples with asymmetric sites are BpmI (CTGGAG 16/14) and BseRI (GAGGAG 10/8). Type IIG enzymes may be Type IIA or Type IIS.

Type IIH restriction endonucleases contain genetic organization similar to Type I restriction-modification system. The DNA recognition sequence can be symmetric or asymmetric. Examples are BcgI (10/12 CGAN₅TGC 12/10) (SEQ ID NO:43) and Bael (10/15 AC N₄ GTAYC 12/7) (SEQ ID NO:44).

Type IIT restriction endonucleases are heterodimers or tetramers (2× heterodimers). The DNA recognition sequence can be symmetric or asymmetric. Examples are Bpu10I (CCTNAGC −5/−2), BbvCI (CCTCAGC −5/−2), and BslI (CCNNNNN/NNGG).

There are over 200 restriction endonucleases described in Rebase®. Of these, there are more than 79 Type IIA restriction enzymes with asymmetric recognition sequences and unique specificity (http://rebase.neb.com/rebase/). Many of these restriction-modification systems have been cloned and expressed in heterologous hosts (Rebase®). It would be desirable to design a general method of engineering nicking endonucleases from restriction enzymes having an asymmetric recognition site to form sequence-specific and strand-specific nicking enzymes.

### SUMMARY

In an embodiment of the invention, a method is provided for engineering a strand-specific nicking endonuclease, that includes the steps of: (a) transforming a first host cell population lacking methylase protection, with plasmids containing a randomly mutagenized restriction endonuclease gene; (b) culturing the transformed host cells of step (a) and isolating the plasmids therefrom; (c) cleaving into fragments the mutagenized restriction endonuclease gene of step (b) and a corresponding wild-type restriction endonuclease gene; (d) performing an in vitro backcross between the wild-type and mutagenized restriction endonuclease fragments of step (c) and obtaining a ligated gene; (e) detecting a strand-specific nicking activity of a protein expressed by the ligated gene of step (d); and (f) identifying the engineered strand-specific nicking endonuclease.

The engineered strand-specific nicking endonuclease may nick dsDNA only or may nick DNA/RNA hybrids on one or both strands or may nick RNA/RNA duplexes. The method may utilize a heterogeneous mixture of plasmids or a homogeneous mixture of plasmids, any of which containing one or more different types of restriction endonuclease or subunits of restriction endonucleases. The mutagenized fragments could be cleaved into two or more fragments. The backcross should however generate after ligation a gene capable of expressing a protein.

In step (c) above, cleaving the gene may be achieved by means of restriction endonuclease digestion where the restriction endonuclease gene fragments are purified on an agarose gel. In step (d) above, a second population of host cells is transformed with the ligated gene wherein the transformants (the transformed host cells) are protected from the adverse effects of expressed enzyme induced cleavage because of methylation by cognate or non-cognate methylases. The method may further include the step of cloning the transformants. These colonies may be individually screened for nicking activity using a supercoiled DNA substrate. Screening can be achieved by utilizing total cells in a culture media or a cell extract.

In an embodiment of the method, the position and type of mutation in the DNA encoding the nicking endonuclease is determined. This mutation may be a deletion, an insertion or a substitution of one or more nucleotides. For example, the mutagenized gene may have a deletion of as few as 3 nucleotides or as many as 600 nucleotides or any number in between. Moreover, the mutagenized gene may include one or a plurality of mutations. The mutations may be clustered at the C-terminal end of the expressed protein or at the N-terminal end or both. Moreover, the mutations may be spread throughout the gene. The identified mutation or mutations may then be introduced by site-directed mutagenesis into an isoschizomer or neoschisomer of the restriction endonuclease. (The amino acid identity of the isochizomer or neoschizomer with the restriction endonuclease may be in the range of 15%-99%.) An additional mutation may be added to the nicking endonuclease described above either by a second cycle of random mutagenesis or by site-directed mutagenesis to enhance the properties of the nicking endonuclease. For example, it may be desirable to enhance the nicking activity, minimize double strand cleavage activity and/or increase the strand specificity.

The host cells referred to in the above method may be prokaryotic or eukaryotic cells. Where prokaryotic cells are utilized, the host cell may include gram positive or gram negative cells for example, E. coli cells or Bacillus strains.

In an embodiment of the invention, the nicking endonuclease is a thermophilic nicking endonuclease. For example, the nicking endonuclease may be derived from BsaI, BsmAI or BsmBI.

In an embodiment of the invention, the duplex DNA strand specificity of the nicking endonuclease is determined. For example, the nicking endonuclease may be a top strand nicking endonuclease or a bottom strand nicking endonuclease.

In an embodiment of the invention, a method is provided for introducing one or more site specific nicks into pre-selected strands of a DNA duplex, the method comprising digesting the DNA duplex with a nicking endonuclease made according to a method described above under conditions for permitting nicking activity.

In an embodiment of the invention, a method is provided for amplifying a target sequence that includes: (a) providing a single-stranded nucleic acid fragment containing the target sequence, the fragment having a 5′ end and a 3′ end; (b) binding an amplification primer for strand-dependent amplification (SDA) to the 3′ end of the fragment such that the primer forms a 5′ single-stranded overhang, the amplification primer comprising a recognition/cleavage site for a synthetic nicking endonuclease made according to a method described above; (c) extending the amplification primer on the fragment in the absence of a derivatized or substituted deoxynucleoside triphosphate and in the presence of a DNA polymerase having strand-displacing activity and lacking 5′-3′ exonuclease activity and in the presence of four deoxynucleoside triphosphates; (d) nicking the amplified double-stranded target sequence with the nicking endonuclease extending from the nick using the DNA polymerase, thereby displacing the first newly synthesized strand from the fragment and generating a second extension product comprising a second newly synthesized strand; and (e) repeating the nicking, extending and displacing steps such that the target sequence is amplified.

In an embodiment of the invention, a method is provided for engineering an enzyme with a modified substrate specificity and activity that includes: (a) forming a randomly mutagenized DNA library wherein the library has one or more genes encoding whole or part of a mutant enzyme, the mutant enzyme being substantially inactive, the substantially inactive enzyme having an N-terminal end and a C-terminal end, wherein the inactivation results from a mutation in the N-terminal end or C-terminal end of a wild-type enzyme; (b) cleaving the one or more genes expressing the inactive endonuclease into at least a first fragment and a second fragment, wherein first fragment encodes the C-terminal end of the enzyme and the second fragment encodes the N-terminal end of the enzyme; (c) performing a ligation between fragments selected from: the first fragment and a third fragment encoding an N-terminal end of the wild type enzyme; the second fragment with a fourth fragment encoding the C-terminal end of the wild type enzyme; or both first and second fragments to third and fourth fragments respectively; and (d) expressing the ligated DNA in a host cell.

In an embodiment of the invention, a method is provided for amplifying a target nucleic acid, that includes (a) nicking at least one strand of a double-stranded target nucleic acid at a plurality of sites with a nicking enzyme made according to claim 1 to form at least two new 3′ termini; (b) extending one or more of the at least two new 3′ termini with a DNA polymerase; (c) nicking the extension product of step (b); and (d) extending the nicking product of step (c) to amplify at least a portion of one strand of the target nucleic acid.

In an embodiment of the invention, a method for rapidly screening nicking enzyme variants using host cells plus culturing media in a DNA nicking reaction is provided containing a supercoiled DNA substrate.

### BRIEF DESCRIPTION OF THE DRAWINGS

FIG. 1 shows a flow diagram of a preferred embodiment of a protein engineering process for isolating site-specific and strand-specific DNA nicking enzymes.

FIG. 2 shows an assay for screening DNA nicking variants using cell cultures derived from overnight cultures of the mutants. 40 isolates (1-40) were screened by agarose gel electrophoresis.

Isolates #11 and #26 (lanes 11 and 26) show nicking activity. Isolates #36 and #40 (lanes 36 and 40) show both nicking and dsDNA cleavage activity. N, nicked circular DNA; L, linear DNA; S, supercoiled DNA.

FIG. 3 shows results of screening isolates for nicking activity analyzed by agarose gel electrophoresis.

FIG. 3A: DNA nicking activity of Nt.BsaI (K150R/R236G) on supercoiled DNA. Nt indicates nicking of top strand. N, nicked circular DNA; L, linear DNA; S, supercoiled DNA. Lane 1, 1 kb DNA size marker; lanes 2, undiluted cell extract; lanes 3 to 10, 2-fold serial dilutions of cell extracts; lane 11, supercoiled pUC19 DNA.

FIG. 3B: DNA nicking activity of Nt.BsaI (R236D) on supercoiled DNA. Lane 1, 1 kb DNA size marker; lane 2, undiluted cell extract; lanes 3 to 10, 2-fold serial dilution of cell extract; lane 11, pUC19 substrate; lane 12, N.BstNBI. N, nicked circular DNA; L, linear DNA; S, supercoiled DNA.

FIG. 4 shows how the DNA nick site can be determined by using run-off sequencing to determine the nicking site of Nt.BsaI (R236D).

| | |
|---|---|
| 5′ GGTCTCN\hat{ }NNNN 3′ | (SEQ ID NO:1) |
| 3′ CCAGAGNNNNN 5′ | |

The nicked circular DNA product was gel-purified and subjected to run-off sequencing. Taq DNA polymerase adds an adenine (A) base at the end of DNA (template-independent DNA transferase activity).

FIG. 5 shows the results of screening isolates for nicking activity.

FIG. 5A: DNA nicking activity of Nb.BsaI on supercoiled DNA. Nb indicates nicking of bottom strand. Lane 1, 1 kb DNA size marker; lanes 2 to 9, 2 fold serial dilutions from 4 to 512-times dilution of cell extract; lane 10, N.BstNBI; lane 11, pUC19 DNA; lane 12, BsaI digested DNA. N, nicked circular DNA; L, linear DNA; S, supercoiled DNA.

FIG. 5B: DNA nicking assays with 10-fold serial dilution of cell extract. Lane 1, 1 kb DNA size marker; Lanes 2-6, BsaI variant N441D/R442G; lanes 7-11, BsaI D(446-544); lanes 12-16, BsaI D(440-544); Lanes 17-19, extracts prepared from cells carrying pUC19. Lane 20, BsaI-digested pUC19. Lanes 1, 7, 12, and 17, undiluted cell extracts; Lanes 3-6,8-11, 13-16, 18-19, 10-fold serial dilution of cell extracts.

FIG. 6 shows how run-off sequencing was used to determine the nicking site of Nb.BsaI.

| | |
|---|---|
| 5′ GGTCTCNNNNN 3′ | (SEQ ID NO:1) |
| 3′ CCAGAGNNNNN\hat{ } 5′ | |

The nicked circular DNA was gel-purified and subjected to run-off sequencing. Taq DNA polymerase adds an adenine (A) base at the end of DNA (template-independent DNA transferase activity).

FIG. 7 shows a schematic diagram of the locations of amino acid substitutions in BsaI that resulted in DNA nicking activity. * indicates the location of amino acid substitutions (not drawn to scale). The full-length BsaI endonuclease contains 544 amino acid residues. D indicates deletions.

FIG. 8 shows the amino acid sequence alignment between BsaI and BsmBI restriction endonucleases using the “Bestfit” of GCG software. The critical Arg (R) residues are shown in bold and underlined. Identical amino acid residues between the two proteins are indicated by a straight line. Amino acid residues with similar properties are indicated by 1 or 2 dots. Top sequence=BsmBI amino acid sequence (SEQ ID NO:31). Bottom sequence=BsaI amino acid sequence (SEQ ID NO:32).

FIG. 9 shows the results of screening isolates for nicking activity by agarose gel electrophoresis

FIG. 9A: DNA nicking activity of Nb.BsmBI (R438D). Lane 1, DNA size marker; lane 2, 1×cell extract; lanes 3 to 9, 2-fold serial dilution of cell extracts (1/2, 1/4, 1/8, 1/16, 1/32, 1/64, and 1/128); lane 10, pBR322 DNA; lane 11, pBR322 digested with BsmBI; lane 12, pBR322 nicked by N.BstNBI. N, nicked circular DNA; L, linear DNA; S, supercoiled.

FIG. 9B: DNA nicking activity of Nt.BsmBI (R233D). Lane 1, DNA size marker; lane 2, 1× cell extract; lanes 3 to 9, 2-fold serial dilution of cell extracts (1/2, 1/4, 1/8, 1/16, 1/32, 1/64, and 1/128); lane 10, pBR322 DNA; lane 11, pBR322 digested with BsmBI; lane 12, pBR322 nicked by N.BstNBI. N, nicked circular DNA; L, linear DNA; S, supercoiled DNA.

FIG. 10 shows how run-off sequencing can be used to determine the nicking site of Nt.BsmBI.

Nt.BsmBI nicking site:

| | |
|---|---|
| 5′ CGTCTCN\hat{ }NNNN 3′ | (SEQ ID NO:2) |
| 3′ GCAGAGNNNNN 5′ | |

FIG. 11 shows how run-off sequencing can be used to determine the nicking site of Nb.BsmBI.

Nb.BsmBI nicking site:

| | |
|---|---|
| 5′ CGTCTCNNNNN 3′ | (SEQ ID NO:2) |
| 3′ GCAGAGNNNNN\hat{ } 5′ | |

FIG. 12 shows the amino acid sequence alignment between BsaI and BsmAI restriction endonucleases using the “Bestfit” of GCG software. The critical Arg (R) residues are shown in bold and underlined. Identical aa residues between the two proteins are indicated by a straight line. Amino acid residues with similar properties are indicated by 1 or 2 dots. Top sequence=BsmAI aa sequence (SEQ ID NO:37). Bottom sequence=BsaI aa sequence (SEQ ID NO:37).

FIG. 13 shows the results of screening isolates for nicking activity using agarose gel electrophoresis FIG. 13A: DNA nicking activity of Nt.BsmAI (R221D). Lane 1, 1 kb DNA size marker; Lane 2, 3 ml of cell extract; Lane 3-9, 3 ml of 2-fold serial dilution of cell extracts; Lane 10, uncut DNA substrate pBR322; Lane 11, BsmAI digestion; Lane 12, N.BstNBI.

FIG. 13B: DNA nicking activity of Nb.BsmAI (N415D/R416G). Lane 1, 1 kb DNA size marker; Lane 2, 3 ml of cell extract; Lane 3-9, 3 ml of 2-fold serial dilution of cell extracts; Lane 10, uncut DNA substrate pBR322; Lane 11, BsmAI digestion; Lane 12, N.BstNBI.

FIG. 14 shows how run-off sequencing can be used to determine the nicking site of Nt.BsmAI (R221D).

Nt.BsmAI nicking site:

| | |
|---|---|
| 5′ GTCTCN\hat{ }NNNN 3′ | (SEQ ID NO:3) |
| 3′ CAGAGNNNNN 5′ | |

FIG. 15 shows how run-off sequencing was used to determine the nicking site of Nb.BsmAI.

Nb.BsmAI nicking site:

| | |
|---|---|
| 5′ GTCTCNNNNN 3′ | (SEQ ID NO :3) |
| 3′ CAGAGNNNNN\hat{ } 5′ | |

FIG. 16 shows the DNA nicking activity of N.BsmAI variants isolated by random mutagenesis and the genetic screen analyzed by agarose gel electrophoresis. Lanes 2-4, N.BsmAI isolate #40, 1×, 1/10, 1/100 diluted cell extract; lane 5-7, N.BsmAI isolate #48, 1×, 1/10, 1/100 diluted cell extract; lanes 8-10, N.BsmAI isolate #55, 1×, 1/10, 1/100 diluted cell extract; lanes 11-13, N.BsmAI isolate #101, 1×, 1/10, 1/100 diluted cell extract; lanes 14-16, N.BsmAI isolate #197, 1×, 1/10, 1/100 diluted cell extract; lane 17, pBR322 DNA; lane 18, BsmAI-digested pBR322; lane 19, pBR322 nicked by N.BstNBI.

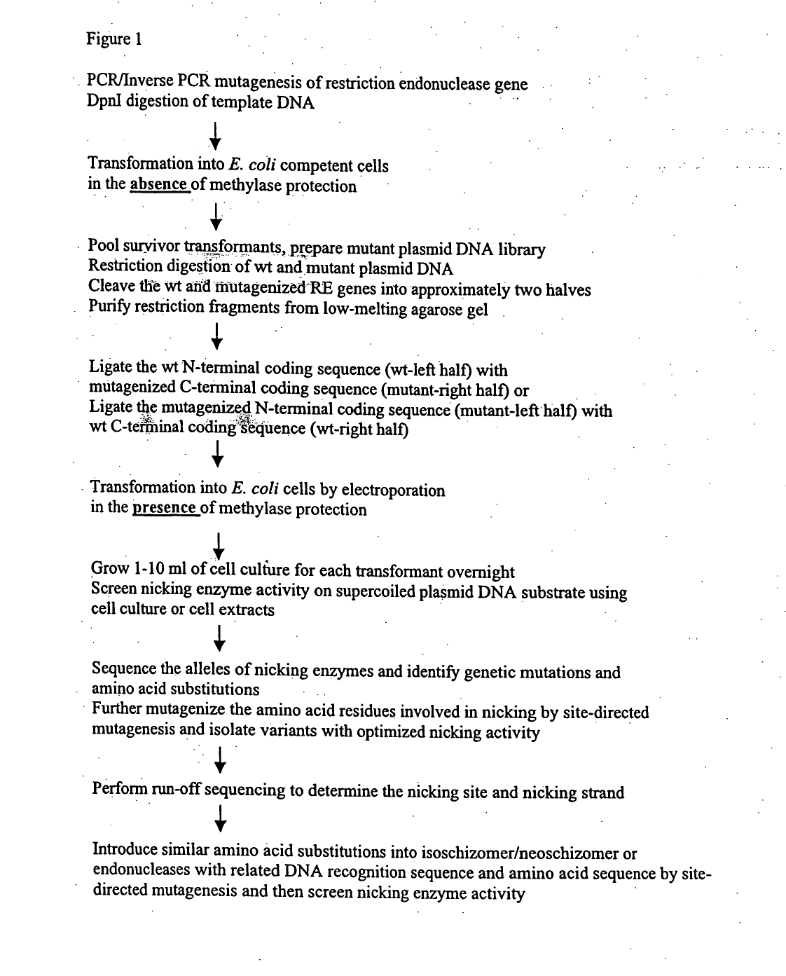

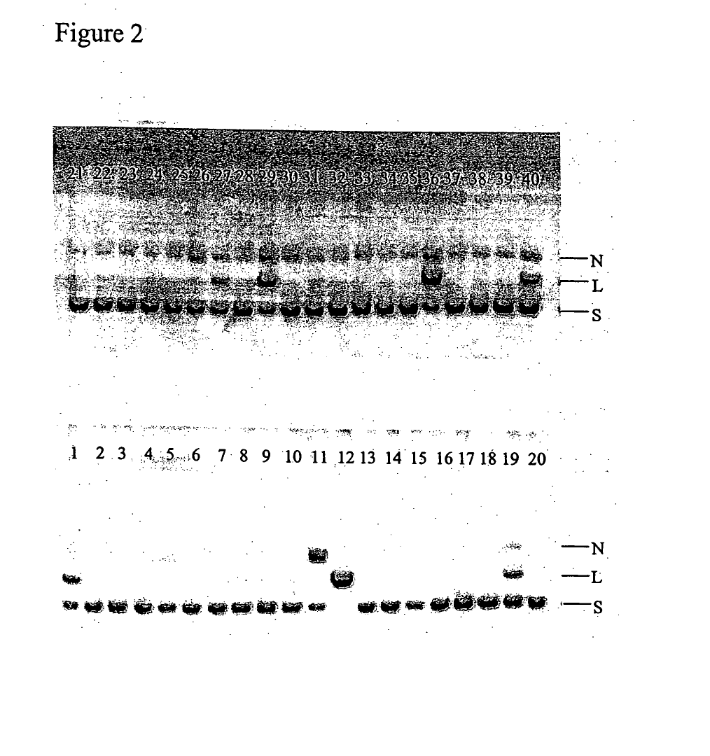

### DETAILED DESCRIPTION OF THE INVENTION

The engineering strategy described herein involves random mutagenesis of a restriction endonuclease gene and construction of a mutant library. The strategy is exemplified using a gene encoding a Type IIA/IIS restriction endonucleases with asymmetric recognition sequences. Significantly, the method can produce nicking endonucleases without prior knowledge of protein structure and active sites. No limitation as to a preferred position of a mutation is proposed here for any enzyme as long as the mutations inactivate restriction endonuclease duplex DNA cleavage activity. Restriction endonucleases cleave dsDNA and are toxic to cells, which lack protective methylation at the cleavage sites of the enzyme. Consequently, when a library of mutated restriction endonuclease genes is transformed into a host lacking protective modification of its DNA, cells transformed with DNA encoding active restriction endonucleases do not survive and consequently, all residual genes encoding highly active alleles are purged from the mutant pool.

Mutant pools and wild-type pools of endonuclease genes are then subjected to restriction digestion of the endonuclease gene to yield fragments. Although it is within the scope of embodiments of the invention to cleave the mutant or wild-type endonuclease gene into more than two fragments, it is preferable to cleave the endonuclease gene into two fragments. The gene could be digested with a restriction endonuclease and split into two parts where the percentage of each of the parts can vary in the range of 5/95 to 50/50 to 95/5, for example, 50/50, 55/45, 60/40, 65/35 or 70/30.

The location of the cleavage site of mutant and wild-type genes may vary from a central position in which two fragments of approximately equal size are formed to an asymmetric position yielding two fragments of substantially different size. However, it is preferable to preserve nicking or binding specificity domains within a fragment. This may result in a preference for a central position for cleavage rather than a peripheral position.

A mutant fragment is then recombined or ligated with the wild-type fragment to generate a gene encoding a functional nicking endonuclease. One benefit of creating a pool of wild-type and mutagenized fragments is that of diluting the effect of mutations on endonuclease activity and permitting endonuclease reactivation. In a preferred embodiment of the method, an in vitro recombination step follows random mutagenesis of the restriction endonuclease gene in which between approximately one-half of the mutagenized (inactivated) allele and the wild-type (wt) coding sequence are recombined in vitro by restriction fragment swapping and ligation.

A host cell protected by modification is transformed with the ligated DNA preferably contained within a plasmid or vector. Significantly, it is here demonstrated that a whole cell culture (cells plus culture media) or cell lysates can be screened for DNA nicking activity from individual transformants to yield candidates of interest. The mutant alleles may be sequenced to identify genetic mutations and amino acid substitutions. In an additional step, variants with optimized nicking activity can be constructed by site-directed mutagenesis of those residues identified by previous screening steps.

A hybrid restriction endonuclease is selected that is capable of substantially cleaving at a specific site and/or on a specific strand. (“Substantially” is here intended to mean “predominantly.”) It is not necessary for purposes of nicking that 100% of one strand of a DNA duplex be cleaved nor that 0% of the second strand be cleaved. However, it is desirable to achieve as high a percentage of nicking as possible such as 95% or higher).

The following terms are described with respect to their use in preferred embodiments of the invention.

The term “synthetic nicking endonuclease” refers to a modified recombinant nicking endonuclease that is a product of fragment exchange between DNA encoding a wild type and a mutagenized restriction endonuclease. The synthetic nicking endonuclease differs from a naturally occurring nicking counterpart by at least one amino acid. The synthetic nicking endonuclease can be further distinguished from a nicking endonuclease formed according to the method described in U.S. patent application 2003-148275 by a random mutation at the C-terminus of the enzyme, more particularly, a deletion mutation at the C-terminus or intermediate region.

The term “C-terminal end” of a protein refers to the half of the protein sequence starting at the ultimate C-terminal amino acid. Similarly, the N-terminal end refers to the half of the protein sequence starting at the ultimate N-terminal amino acid.

The term “mutation” refers to a deletion, an insertion or a substitution of amino acids. The mutation may involve one or more amino acids at a particular site in the protein and additionally may encompass multiple sites.

The term “gene” refers to a nucleic acid sequence that encodes a protein or peptide.

The term “restriction endonuclease” is intended to encompass active, in active and wild-type enzymes where appropriate in the context.

The term “inactivated” with respect to restriction endonucleases refers to restriction endonucleases that are not toxic to host cells containing unmodified DNA.

A preferred embodiment of a method for creating nicking endonucleases is provided below.

1. A mutant endonuclease library is constructed by random mutagenesis of the restriction endonuclease gene using, for example, error-prone inverse PCR. Mutagenized DNA is transformed into E. coli in the absence of protecting methylases. The DNA from the survivor colonies is pooled and amplified to form a mutant plasmid library with inactivated endonuclease genes. The cloned endonuclease genes contained in the survivors are functionally disabled, as deduced from survival in the absence of protective methylation.

2. An in vitro backcross is performed with the wild-type gene, in which the fragments from the mutagenized pool are substituted for the corresponding wild-type segment of the gene. This may be achieved by: separating the mutagenized gene into two fragments by restriction digestions; gel-purifying the restriction fragments; ligating the mutagenized N-terminal coding sequence to the wild-type C-terminal coding sequence or ligating the wild-type N-terminal coding sequence to the mutagenized C-terminal coding sequence; and transforming the ligated DNA into E. coli host in the presence of protecting methylases (cognate or non-cognate methylase).

3. A screening assay to detect nicking endonucleases is performed. For example, nicking enzyme activity from cell culture or cell extracts of individual transformants can be tested on appropriate DNA substrates such as supercoiled DNA.

4. Optimization of nicking enzyme activity can be achieved by site-directed mutagenesis of the amino acid residues identified in (3). Variants containing different amino acid substitutions that display optimized nicking enzyme activity and minimal double strand DNA cleavage activity can be obtained. Amino acid residues may be identified for related endonucleases which when subjected to targeted mutagenesis at a predetermined position, affect nicking activity and create a superior nicking enzyme.

The amino acid substitutions affecting nicking activity identified in (3) and (4) above can be introduced into isoschizomers, neoschizomers, or enzymes with related recognition sequence and similar amino acid sequence composition.

The methods described above were used for generating a nicking endonuclease from Type IIS restriction endonuclease BsaI (Example 1). BsaI variants were isolated and amino acid substitutions identified. These variants were found to have nicking activity that predominantly nick the top or the bottom strand of a DNA duplex (top and bottom strand nicking enzymes were named Nt.BsaI and Nb.BsaI, respectively).

The corresponding amino acid substitutions were then introduced into Type IIS restriction endonucleases BsmBI and BsmAI, which have similar DNA recognition sequences and also have similar amino acid sequences. In Example 2, BsmBI nicking enzymes were isolated that predominantly nick the top or the bottom strand (top and bottom strand nicking enzymes were named Nt.BsmBI and Nb.BsmBI). Similarly, Nt.BsmAI and Nb.BsmAI nicking enzymes were constructed by site-directed mutagenesis based on the mutation information derived from N.BsaI nicking variants. Additionally in Example 3, N.BsmAI variants were isolated using the random mutagenesis and the genetic screening method as described herein.

Random Mutagenesis

Random mutagenesis can be achieved by any method known in the art (Heitman, J. and Model, P., EMBO J. 9:3369-3378 (1990)). For example, a restriction endonuclease gene can be mutagenized by chemical mutagen treatment (sodium bisulfite, NH₂OH, nitrosoguanidine et al.), or by passing through a mutator strain (MutD⁻/MutH⁻/MutS⁻, deficient in mismatch DNA repair), or by exposure of cells carrying the target gene to UV or g radiation, or by passing through a strain with a proof-reading deficient DNA polymerase. Alternatively, one skilled in the art can perform Ala scanning to change (presumed) important residues such as Asp, Glu, Lys, Arg residues in a Type IIA/IIS endonuclease protein to Ala and then screen DNA nicking activity in mutant cell extracts. One can also mutagenize the target gene by inverse PCR, error-prone PCR or other PCR based methods of mutagenesis alone or in combination, ligate the PCR product to a cloning vector, and then transform the ligated DNA into a host to construct a mutant plasmid DNA library.

Cloning the Mutagenized DNA and Analyzing Expression

Following the PCR mutagenesis step, the mutagenized plasmid DNA may optionally be digested with DpnI which digests the Dam-methylated template DNA. The DNA is then transformed into E. coli expression host in the absence of cognate or non-cognate methylase protection. Active mutants with dsDNA cleavage activity are eliminated because they kill the host due to extensive DNA damage. The low activity mutants, null mutants, nicking mutants, mutants deficient in cleavage and proficient in binding are among the survivor transformants. All the transformants are pooled and amplified and plasmid DNA is prepared to form the mutant plasmid DNA library. Alternatively, one can screen individual transformants for nicking activity in cell culture or cell extracts with a high through-put screening system.

Cleavage of a Mutacenized Gene and Recombination with a Cleaved Wild-Type Gene Fragment

The mutagenized gene may be split into two or more fragments and backcrossed. One way to do this is by restriction fragment exchange and another way would be by PCR. Plasmid DNA is then digested with two restriction endonucleases, one enzyme cleaves within the endonuclease gene to split the gene into the desired fragments and the other enzyme cleaves in the vector (preferentially in the plasmid replication region). The same enzymes also cleave the wild-type (wt) plasmid into fragments. The cleavage site in the restriction gene does not have to be exactly in the middle of the endonuclease gene. The exact cleavage point depends on the available restriction sites within the gene. Following restriction digestion, the restriction fragments are gel-purified from a low-melting agarose gel. The wt N-terminal coding sequence is combined with the mutagenized C-terminal coding sequence. Conversely, the mutagenized N-terminal coding sequence is combined with the wt C-terminal coding sequence. The DNA fragments are ligated and transferred into an expression host in the presence of protective methylation conferred by a cognate or non-cognate methylase. If the nicking enzyme activity is not lethal to the host or the nicking enzyme expression is under very tight control (repressed) the methylase protection may not be required. Electroporation is the preferred method for transformation. However, high efficiency chemically competent cells can also be used for transformation.

An alternative method for the backcrosses with the wt coding sequence is to perform localized random mutagenesis and then ligate the mutagenized DNA fragment to substitute the wt restriction fragment. In addition, one can mutagenize a portion of the coding sequence by error-prone PCR and then ligate the mutagenized fragment to the rest of the wt DNA fragment.

Assaying Nicking Activity

Individual transformants may be cultured in a small culture (for example, 1-10 mls) overnight. The cell cultures include appropriate antibiotics for plasmid selection. Whole cell cultures (cells plus media) are directly used to assay the nicking activity of individual mutant on supercoiled DNA substrates. Some E. coli cells are lysed and enzymes are released into the assay reactions. Alternatively, the cell pellet can be resuspended in a sonication buffer or lysis buffer. Cells can be lysed by sonication, lysozyme treatment, detergent treatment, or freeze-thawing. Once DNA nicking activity is detected in the cell culture, the nicking activity can be further confirmed using sonicated and clarified cell extracts.

Sequencing the Mutant Allele

Once the nicking enzyme activity is confirmed, the entire coding sequence of the mutant allele can be sequenced, for example, by dideoxy terminator sequencing. The genetic mutation and amino acid substitutions are then identified. If multiple amino acid substitutions are found in the same protein, the individual amino acid changes can be segregated by site-directed mutagenesis and each single mutant may be evaluated for DNA nicking activity.

Optimization of Variants with Nicking Activity

In some cases, variants with DNA nicking activity also show weak dsDNA cleavage. In order to optimize the nicking activity, the identified amino acid (aa) residue may be subjected to site-directed saturation mutagenesis to isolate variants each of which contain one or more of the remaining 18 aa substitutions. The DNA nicking activity is then compared among all the mutants. The best nicking variant with minimal dsDNA cleavage activity is then selected. In some cases, two or more aa substitutions are required for optimal nicking activity and maximum specific activity. In other cases, truncation mutants (C-terminal deletion mutants) also display DNA nicking activity. It is also likely that two aa substitutions with partial effects may be first identified in two different mutants. When the two aa substitutions are combined into one mutant, the combined mutations may result in a superior nicking enzyme.

The nicking enzyme is partially purified by column chromatography (e.g. affinity column, ion-exchange column) and then used to nick appropriate supercoiled DNA substrate. The nicked DNA is subjected to run-off sequencing to determine the nicking strand. To facilitate protein purification, protein (peptide) tags such as His tags, chitin-binding domain tags or maltose-binding domain tags can be added to the C-terminus of the nicking enzymes. The fusion protein can be purified by binding to affinity columns and the tags can be removed by protease treatment or protein splicing.

Host Cells

The bacterial host for screening nicking enzyme variants is not limited to E. coli. Bacterial hosts such as Bacillus and Pseudomonas can also be used with appropriate cloning/expression vectors to obtain a reasonable efficiency of DNA transformation or electroporation. For thermophilic enzymes, the Thermus thermophilus host and Thermus-E. coli shuttle vector can be used (Wayne, J. and Xu, S.-y., Gene 195:321-328 (1997)). Genetic screening methods for isolating nicking enzymes can be applied to other DNA cleaving enzymes such as phage terminase, transposase, recombinase, integrase, intron-encoded endonuclease or intein-encoded endonuclease.

The preferred method described herein by which a Type IIA/IIS restriction endonuclease, BsaI, was converted to strand-specific nicking enzymes included the following steps:

1. The bsaIR gene (pUC-BsaIR) was mutagenized by error-prone inverse PCR (25 cycles). The PCR product was digested with XhoI and DpnI and self-ligated. The ligated DNA was transferred into E. coli ER2566 competent cells by transformation. Approximately 2,926 survivor transformants were obtained in the mutant library. Mutant plasmid DNA was prepared by Qiagen columns.

2. The mutagenized plasmids and the wt plasmid were digested with PstI and BseYI. PstI is located in the middle of bsaIR gene and BseYI is within the pUC ColE1 replication origin. Two restriction fragments were gel-purified and swapped with the respective wt fragments, ligated and transferred into E. coli expression host by electroporation. The recipient host carried BsmAI methylase (M.BsmAI) for host DNA protection.

3. Five ml overnight cell cultures were made for individual transformants in LB plus Ap and Cm in a 37° C. shaker. Cells were pelleted by low speed centrifugation. Ten ml of cell culture was used to nick (digest) 1 mg of supercoiled pUC19 DNA. After DNA nicking activity was found in the cell culture, the cell pellet was resuspended in a sonication buffer and lysed by sonication. The lysate was clarified and then used to confirm the nicking enzyme activity in cell extract. A total of 271 mutants were screened for DNA nicking activity. Five mutants displayed nicking activity in both cell culture and cell extracts. The bsaIR* alleles in these five nicking variants were sequenced. Some false positives were also found in the screen. Initially, nicking activity was detected in cell culture, but no apparent nicking activity was detected in cell extracts. Those false positives were discarded and not examined further.

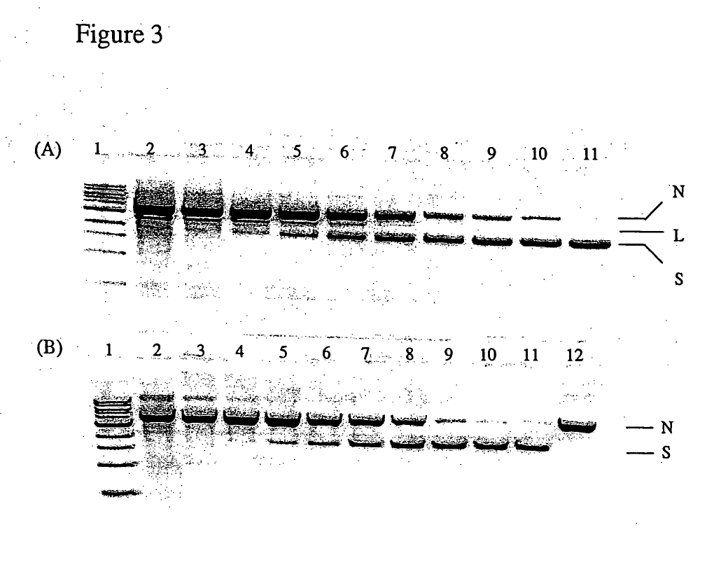

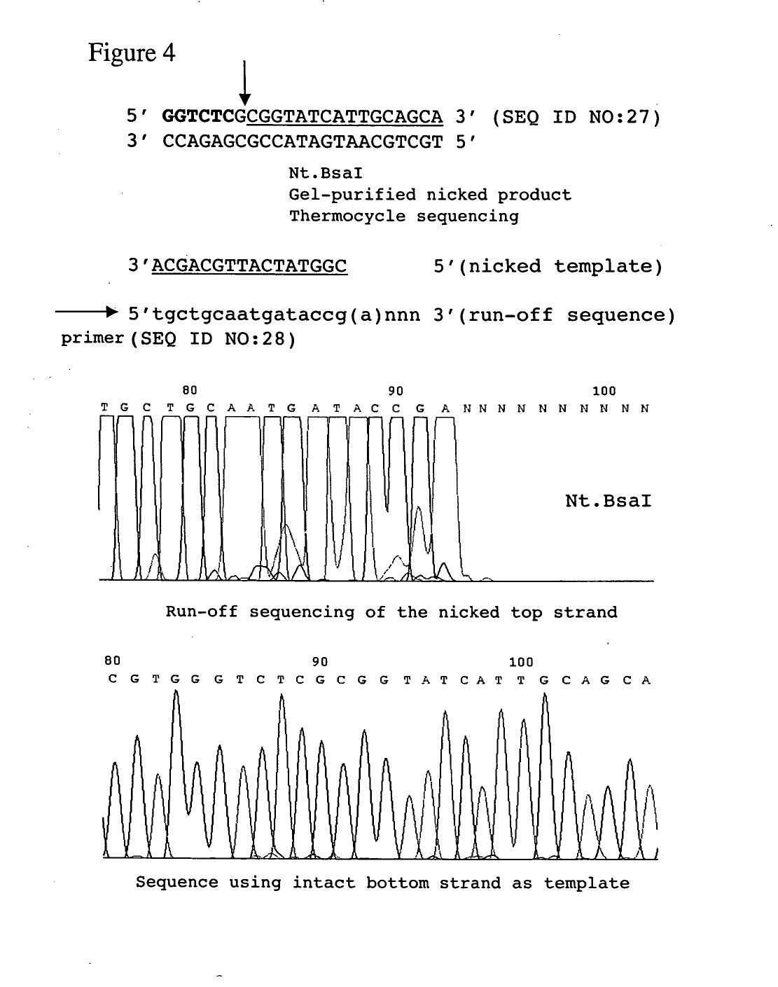

4. Nicking enzyme variant B3 carried aa substitutions K150R and R236G. Another nicking variant B36 also carried two aa substitutions S128L and R236G. It was concluded that R236G is the most likely aa change that contributed to the nicking activity. Since double mutants K150R/R236G (FIG. 3A), S128L/R236G, and single mutant R236G show some residual dsDNA cleavage activity (˜10%) in addition to the nicking activity, another single mutant R236D was constructed by site-directed mutagenesis. FIG. 3B shows that BsaI variant R236D predominantly nicks supercoiled DNA. The nicked DNA products were used as the template for run-off sequencing to determine the nicked strand. FIG. 4 shows that it nicks the top strand as indicated by the sharp decrease in sequence signal after the nicked site. Therefore N.BsaI variant R236D was named Nt.BsaI.

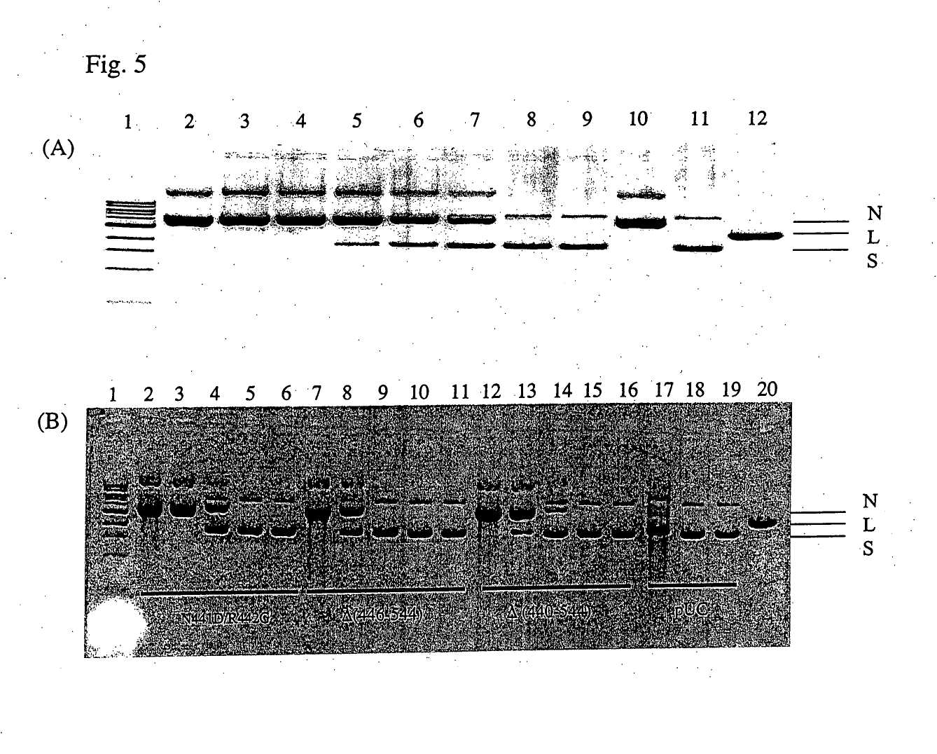

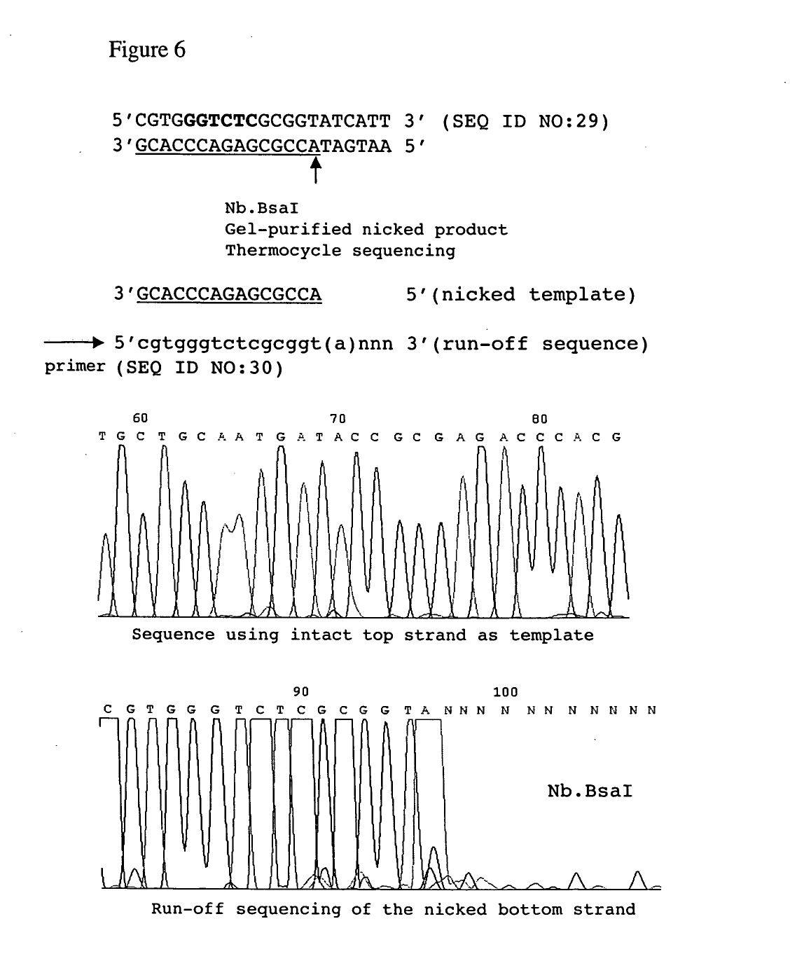

5. Nicking enzyme variant All carried two aa substitutions N441D and R442G. The DNA nicking activity of N441D/R442G was shown in FIG. 5A. The aa changes N441D and R442G were separated by site-directed mutagenesis to construct single mutants carrying either N441D or R442G. The dsDNA cleavage activity of variant N441D is similar to that of the wt enzyme. R442G displayed some dsDNA cleavage activity in addition to nicking. Therefore the two adjacent aa substitutions may act synergistically to achieve the high nicking activity. The nicked DNA product was used as the template for run-off sequencing to determine the strand of nicking. FIG. 6 show that the BsaI variant N441D/R442G nicks the bottom strand and therefore it was named Nb.BsaI. The top strand remains intact and generates normal DNA sequence.

6. During the nicking enzyme screen, it was found that BsaI deletion variants also displayed DNA nicking activity. The two deletion variants resulted from a sense codon mutation to a stop codon. In isolate A26 the Leu446 codon was mutated to a stop codon (UUA to UAA). Therefore the BsaI aa sequence after 446 was deleted in A26 D(446-544). In isolate A57, the Glu440 codon was mutated to a stop codon (GAA to UAA). Therefore the aa sequence after 440 is deleted in mutant A57 D(440-544). Deletion variant A57 introduces nick at the bottom strand as determined by run-off sequencing.

7. The important aa substitutions that are responsible for nicking activity can be introduced into isoschizomer/neoschizomer and other restriction endonucleases provided they share similar DNA recognition sequence or share aa sequence similarity/identity of the proteins. By aa sequence alignment, homologous regions were found among BsaI, BsmAI and BsmBI. The same aa substitutions were introduced into BsmBI and generated Nt.BsmBI and Nb.BsmBI nicking enzymes. When the same aa substitutions were introduced into BsmAI, it generated Nt.BsmAI and Nb.BsmAI nicking enzyme. However, the Nb.BsmAI variant isolated by site-directed mutagenesis of aa residues corresponding to BsaI nicking enzymes also displays dsDNA cleavage activity. In general, the important aa substitutions can be introduced into isoschizomers or neoschizomers when they share significant aa sequence similarity (15% to 99% aa sequence similarity).

Uses for nicking enzymes include:

1) Strand displacement DNA amplification. A specific nick can be introduced in the target DNA by a nicking enzyme. Bst DNA polymerase or other DNA polymerases can initiate a new strand synthesis at the nick and displace the nicked strand, resulting in linear DNA amplification products.

2) Recombinant DNA technology for gene fragment assembly. DNA fragment assembly may by achieved by, for example, introducing staggered nicks in top and bottom strands of a duplex DNA to generate long single strand cohesive ends (8 to 20 nt long). The complementary cohesive ends can anneal together. Because the fragments are so long, it is often possible to bypass the ligation step and the annealed DNA can then be used directly in transformation, electroporation, or transfection. Nicking enzymes can also be used in preparation of single-stranded DNA ends for DNA fragment assembly in linear or circular form.

3) Genetic polymorphism detection. Small PCR fragments can be amplified from genomic DNA. A nick is introduced into the target by a nicking enzyme. After denaturing HPLC, the nicked product can be “read” by mass spectrometry to detect genetic alterations. Nicking enzymes may be used for oncogene mutation detection, bacterial and viral pathogen detections.

4) Duplex DNA containing a single nick exhibits altered migration in a gel mobility assay. This characteristic could be used for studying differential base stacking and nearest-neighbor energetics (Kuhn, H. et. al. Electrophoresis 23:2384-7 (2002)).

5) DNA mismatch excision repair can be studied by preparation of nicked-duplex DNA or gapped DNA (Wang, H. & Hays, 3. B., J Biol Chem 277:26136-42 (2002)).

The present invention is further illustrated by the following Examples. The examples are provided to aid in the understanding of the invention and are not construed as a limitation thereof. It is understood that for the experienced researchers, minor modifications and variations can be made from the present invention and the same outcome can be achieved. The references cited above and below are herein incorporated by reference. Additionally, Zhu et al. J. Mol. Biol. 337, 573-583 (2004) and Samuelson et al. Nucl Acids Res 32, 3661-3671 (2004)) are incorporated by reference.

### EXAMPLES

### Example 1

### Engineering Strand-Specific Nicking Enzymes Nt.BsaI and Nb.BsaI from BsaI Restriction Endonuclease

1. Construction of Mutant Plasmid DNA Library

The bsaIR gene was first cloned into pUC19 to generate pUC-BsaI. The endonuclease gene was sequenced to confirm the wt sequence. The bsaIR gene was mutagenized by four separate reactions of error-prone inverse PCR under the condition of 94° C. for 30 sec, 55° C. for 30 sec, 72° C. for 4 min 8 sec for 25 cycles with Taq DNA polymerase in the presence of 2, 4, 6, or 10 mM MgCl₂, dNTP, pUC-BsaI template, and 1× Thermopol buffer. PCR products were pooled and purified by Qiagen spin columns and digested with XhoI and DpnI. DpnI digestion removed the Dam-methylated template DNA (wild type). The PCR product was self-ligated, transferred into E. coli expression host ER2566 by electroporation and transformants were plated on Ap plates. The recipient host did not carry a protective methyltransferase and therefore wt enzyme or mutant enzymes with high activities killed the host cells. Only null mutants, mutants with low activity, or mutants with cleavage deficiency and binding proficiency survived this transformation step. A total 2936 survivor transformants were obtained and pooled together. Plasmid DNA was prepared from pooled cells to form a mutant plasmid DNA library.

2. Restriction Digestion of wt and Mutant Plasmid DNA, Restriction Fragment Exchange and Ligation

The wt and the mutant plasmids (˜4.1 kb) were digested with restriction enzymes PstI and BseYI. PstI was located in the middle of the bsaIR gene and PstI digestion split the endonuclease gene into two equal segments. BseYI is located in the ColEI replication origin of the vector. The restriction fragments were gel-purified from a low-melting agarose gel and further purified by Qiagen spin columns. The wt N-terminal coding sequence (wt left half) was combined with the mutagenized (inactivated) C-terminal coding sequence (mutant right half) and ligated with T4 DNA ligase. Conversely, the mutagnized (inactivated) N-terminal coding sequence (mutant left half) was combined with the wt C-terminal coding sequence (wt right half) and ligated with T4 DNA ligase. The ligated DNA was transferred into E. coli expression host ER2683 [pACYC-BsmAI] by standard transformation or electroporation. The non-cognate methylase M.BsmAI (recognition sequence GTCTC) modifies all BsaI sites as well as additional related sites and therefore conferred host DNA protection. Standard transformation using chemically competent cells gave rise to few transformants, so electroporation was the preferred method for transformation. Transformants were plated on LB agar plates+Ap and Cm and incubated overnight at 37° C.

3. Screening Nicking Enzyme Variants from Cell Culture and Cell Extracts

Single colonies from the transformation were inoculated into 10 ml of LB+Ap and Cm and cultured overnight in a 37° C. shaker. Ten ml of total cell culture were used to nick (digest) 1 mg of pUC19 supercoiled DNA in a nicking assay. As a positive control, the same substrate was nicked by a strand-specific nicking enzyme N.BstNBI. The nicked (digested) DNA was analyzed by agarose gel electrophoresis. When nicked circular DNA was detected in the culture medium of an isolate, the corresponding cell pellet was thawed, lysed by sonication and clarified by centrifugation. The cell extract was used to confirm the nicking enzyme activity. In some cases, cell extracts need to be diluted by a serial dilution when high nicking enzyme activity was detected. For example, a 2-fold serial dilution was performed from 2-fold to 512-fold dilutions to detect the nicking activity of mutant isolate A11 (Nb.BsaI, N441D/R442G, FIG. 5A). In a first screen of 160 clones, 11 showed positive nicking activity in cell culture and 3 clones (A11, A26, and A57) were later sequenced and characterized. In a second screen, 6 positive nicking activities were detected in the cell cultures and two (B3 and B36) were sequenced and characterized. Those that showed some nicking activity in cell culture and no apparent nicking activity in cell extracts were assumed false positive and were discarded. Those variants that showed some nicking and strong double strand DNA cleavage were also discarded. Only the variants that showed predominantly nicking activity were sequenced and further analyzed. The following primers were used to sequence the entire bsaIR alleles coding for the nicking variants:

S1224S and S1233S (pUC universal forward and reverse primers)

(SEQ ID NO:4)

5′ CGATGTGTCTATTTCGTTCATCCA 3′ (299-313)

(SEQ ID NO:5)

5′ GGCAAGAATTAATAGACTGGATGG 3′ (299-314)

(SEQ ID NO:6)

5′ TTTGAAATTGATTCACTAGAACAT 3′ (299-315)

(SEQ ID NO:7)

5′ AAGGAAACTTGTTTAAATGATAAC 3′ (299-316)

(SEQ ID NO:8)

5′ AGAGATACAAAATACACTGAAGAG 3′ (299-317)

Table 1 summarizes the mutant isolate number, base mutation, aa substitution, and strand of nicking.

TABLE 1
Nicking enzyme variants isolated by random
mutagenesis of bsaIR gene

| | | | Strand of |
|---|---|---|---|
| Isolate number | Base mutation | aa substitution | nicking |
| A11 | AAC to GAC | N441D/R442G | Bottom |
| | AGA to GGA | | |
| A26 | UUA to UAA | Stop codon at | Not determined |
| | | 446 | |
| | | D(446-544) | |
| A57 | GAA to UAA | Stop codon at | Bottom |
| | | 440 | |
| | | D(440-544) | |
| B3 | AAA to AGA | K150R/R236G | Top |
| | AGA to GGA | | |
| B36 | UCG to UUG | S128L/R236G | Top |
| | AGA to GGA | | |

4. Site-Directed Mutagenesis of aa Residues Implicated in Nicking Enzyme Activity

Since both isolate B3 and B36 carry R236G substitutions, it was concluded that Arg236 to Gly substitution is probably the residue that is responsible for the nicking phenotype. We reasoned that conversion of this amino acid (R) to an acidic one (D) might have even stronger nicking activity than the Gly substitution. Asp was chosen because of its negative charge of the side chain. Site-directed mutagenesis (inverse PCR) was carried out to construct single mutant R236G and R236D. The inverse PCR primers have the following sequences:

(SEQ ID NO:9)

R236G forward primer
5′ TCATATACAACAGATAGAGGAGCATTTGAATACTGGGTT 3′

(SEQ ID NO:10)

R236G reverse primer
5′ AACCCAGTATTCAAATGCTCCTCTATCTGTTGTATATGA 3′

(SEQ ID NO:11)

R236D forward primer
5′ TGATATACAACAGATAGAGACGCA11TGAATACTGGGTT 3′

(SEQ ID NO:12)

R236D reverse primer
5′ AACCCAGTATTCAAATGCGTCTCTATCTGTTGTATATGA 3′

Inverse PCR reactions were carried out under the conditions of 94° C. for 30 sec, 55° C. for 30 sec, 72° C. for 4 min 8 sec for 20 cycles with Vent DNA polymerase in the presence of 2, 4, and 8 mM MgCl₂, dNTP, pUC-BsaIR template, and 1× Thermopol buffer. Amplified products were digested with DpnI to destroy the template DNA. Single mutants were screened from transformants and the mutants were sequenced to confirm the desired mutation. Cell extracts were prepared for variants R236G and R236D and assayed for strand-specific nicking activity. It was found that R236D displays predominant nicking activity and very little dsDNA cleavage activity (FIG. 3B). Variants K150R/R236G (FIG. 3A), R236G also nicks DNA, but a small fraction of DNA suffered double-stranded cleavage. It was concluded that Arg236 to Glu or Gly substitution is responsible for the conversion to nicking enzymes. This experiment demonstrated that after random mutagenesis to identify the aa residues implicated in nicking activity, one can use site-directed mutagenesis to construct more variants with optimized strand-specific nicking activity.

The nicking variant N441D/R442G carries two aa substitutions. The nicking activity of BsaI variant N441D/R442G are shown in FIG. 5A. Site-directed mutagenesis was carried out to construct single mutants N441D and R442G. The inverse PCR primers have the following sequences:

(SEQ ID NO:13)

N441D forward primer
5′ AGCTATATTGAAAAAGAAGACAGAAATGCCTTATTAGTAATA 3′

(SEQ ID NO:14)

N441D reverse primer
5′ TATTACTAATAAGGCATTTCTGTCTTCTTTTTCAATATAGCT 3′

(SEQ ID NO:15)

R442G forward primer
5′ AGCTATATTGAAAAAGAAAACGGAAATGCCTTATTAGTAATA 3′

(SEQ ID NO:16)

R442G reverse primer
5′ TATTACTAATAAGGCATTTCCGTTTTCTTTTTCAATATAGCT 3′

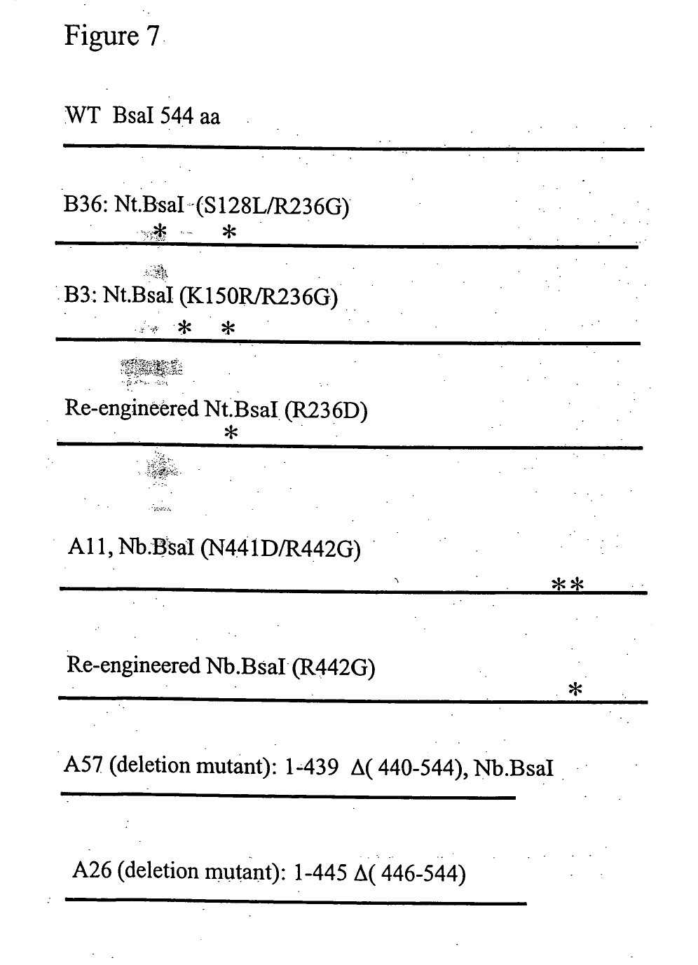

Inverse PCR reactions were carried out under the conditions of 94° C. for 30 sec, 55° C. for 30 sec, 72° C. for 4 min 8 sec for 20 cycles with Vent DNA polymerase in the presence of 2 and 4 mM MgCl₂, dNTP, pUC-BsaIR template, and 1× Thermopol buffer. Amplified products were digested with DpnI to eliminate the template DNA. Mutants were isolated after transformation and the desired mutation was confirmed by DNA sequencing. Variant N441D shows dsDNA cleavage activity whereas variant R442G shows predominant nicking activity. However, the nicking activity of the single mutant R442G is approximately 8-fold lower than the double mutant N441D/R442G. It was estimated from the nicking activity in cell extracts that R442G has 1×10⁵ units/gram of wet cells as opposed to 8×10⁵ units/gram of wet cells for the double mutant N441D/R442G. Therefore, it was concluded that in the double mutant N441D/R442G, the two aa substitutions may act synergistically to convert BsaI to nicking enzyme with high activity. FIG. 7 lists all the BsaI nicking enzyme variants isolated by random and site-directed mutagenesis.

6. Isolation of BsaI Deletion Variants that Display DNA Nicking Activities

As shown in Table 1, two deletion variants A26 and A57 were isolated from the random mutagenized plasmid DNA library. A26 and A57 carried aa deletion from 446 to 544 and from 440 to 554, respectively. The nicking activity of the two deletion variants are shown in FIG. 5B (lanes 7-16). This is the first demonstration that C-terminal deletion mutations can convert a Type IIA/IIS restriction endonuclease into DNA nicking enzyme. The nicked DNA produced by A57 was used as the template for run-off sequencing. It was found that A57 D(440-544) nicks the bottom strand. Therefore it was named Nb.BsaI D(440-544). The nicking activity of Nb.BsaI D(440-544) is at least an order of magnitude lower than nicking variants Nt.BsaI (R236D) or Nb.BsaI (N441D/R442G).

7. Partial Purification of Nicking Enzymes

N.BsaI nicking enzymes were partially purified by chromatography through heparin Sepharose and DEAE Sepharose columns. Proteins were eluted from heparin Sepharose column with a NaCl gradient of 50 mM to 1 M. Proteins passed through DEAE Sepharose column as flow-through and DNA/RNA remained bound to the column.

8. Molecular Biology Techniques Used in this Work

PCR, site-directed mutagenesis inverse PCR procedure: PCR conditions are 94° C. for 30 sec, 55° C. for 30 sec, 72° C. for 4 min 8 sec for 20-25 cycles with Taq or Vent DNA polymerase in the presence of 2-10 mM MgCl₂, dNTP, pUC-BsaI template, and 1× Thermopol buffer. For random mutagenesis, Taq DNA polymerase was used in inverse PCR. For oligonucleotide-directed mutagenesis, Vent DNA polymerase was used in inverse PCR. The template DNA in the amplified DNA was removed by DpnI digestion.

Plasmid DNA preparation procedure: Qiagen spin columns were used to prepare plasmid DNA. Cells lysis, protein and cellular DNA denatuation were performed with the addition of P1, P2, and N3 buffers. Clarified supernatant containing Plasmid DNA was loaded onto Qiagen spin columns and washed with PB and PE buffers. Plasmid DNA was eluted with 10 mM Tris-HCl buffer.

Transformation procedure: Chemically competent cells were prepared by treatment of exponential phase E. coli cells with ice-cold 50 mM CaCl₂ for 30 min. Competent cells were mixed with plasmid DNA and incubated on ice for 30 min. After 3-5 min heat treatment at 37° C., an equal volume of LB broth was added. Cells were regrown in a 37° C. incubator for one h. Transformants were plated on LB agar plates with appropriate antibiotics for plasmid selection.

Electroporation procedure: Electro-competent cells were prepared by washing E. coli exponential phase cells in 10% ice-cold glycerol twice (500 ml 10% glycerol for cell pellet from 1 L cell culture). After mixing the DNA with 100 ml of competent cells electroporation was carried out under the condition of 1900 V, 200 Ω, 25 mF, 0.1 cm cuvette. One ml of LB was added to cells and incubated for 1 h to amplify the transformants. Transformants were plated on LB agar plates with appropriate antibiotics (Ap, Ap plus Cm) for plasmid selection.

Preparation of cell extracts: Cells were cultured overnight in a 37° C. shaker, then pelleted by low speed centrifugation (1800 g). Cells were resuspended in a sonication buffer (50 mM Tri-HCl, pH 7.8, 10 mM b-mercaptoethanol, 50 mM NaCl). Cell lysis was completed with sonication at output 4, 50%, discontinuous burst 5 times with a small sonication tip. The lysate was clarified by centrifugation at 14000 g at 4° C. for 10 min. The supernatant (cell extract) was used for the nicking enzyme assay.

Nicking enzyme assay: 10 ml of cell culture or diluted cell extract was used to nick (digest) 1 mg of supercoiled pUC19 DNA for 1 h in 1×BsaI restriction buffer. Reaction was terminated by addition of stop buffer (1% SDS, 50% glycerol, 0.1% BPB dye). Nicked products were resolved in 0.8 to 1% agarose gels with TBE buffer.

Run-off sequencing and DNA sequencing: Nicked products or gel-purified nicked products were used for run-off sequencing. Big dye AmpliTaq dideoxy terminator sequencing kit was used in the sequencing reactions (Applied Biosystems, Foster City, Calif.). DNA sequence was resolved on automated sequencer ABI373A. DNA sequence was edited and analyzed with Seqed program and GCG programs.

### Example 2

### Engineering BsmBI Nicking Variants from BsmBI Endonuclease by Site-Directed Mutagenesis of aa Residues Corresponding to the aa Substitutions in Nt.BsaI and Nb.BsaI

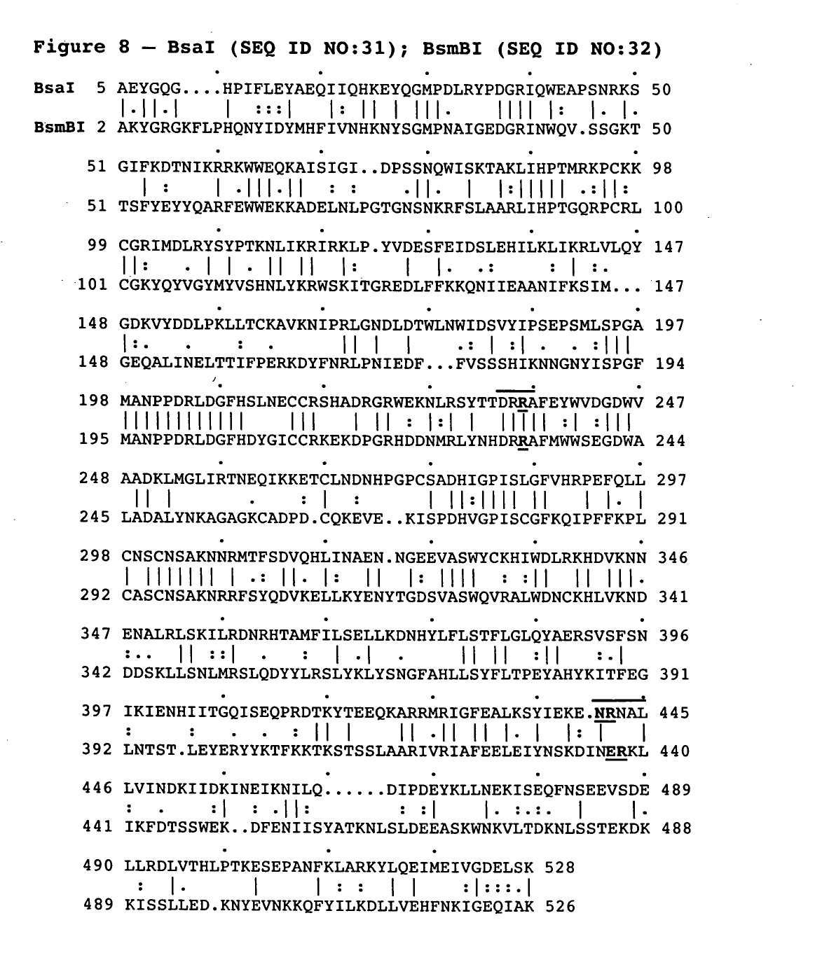

The BsaI and BsmBI restriction endonucleases recognize similar DNA recognition sequence (BsaI GGTCTC; BsmBI CGTCTC) and cleave N1/N5 downstream. The two endonucleases show 45% similarity and 34% sequence identity at the amino acid level (FIG. 8). It was reasoned that the aa substitutions identified in BsaI that converted BsaI endonuclease to nicking enzymes could be directly introduced into the BsmBI restriction endonuclease by site-directed mutagenesis to yield BsmBI nicking variants. The prerequisite for such equivalent mutation transfer is that the two proteins share similarity in aa sequence, active sites, and structure. The aa sequence alignment program “Bestfit” of GCG shows that the residue Arg233 of BsmBI corresponds to the Arg236 of BsaI (FIG. 8). As demonstrated in Exampe 1, Arg236 to Gly or Arg236 to Asp in BsaI endonuclease generated BsaI nicking variants (Nt.BsaI (R236D), Nt.BsaI (R236G)). A corresponding BsmBI variant R233D was constructed by oligonucleotide-directed mutagenesis (inverse PCR). The inverse PCR primers have the following sequences:

(SEQ ID NO:17)

R233D forward primer
5′ CTATATAATCATGATAGAGATGCTTTTATGTGGTGGTCA 3′

(SEQ ID NO:18)

R233D reverse primer
5′ TGACCACCACATAAAAGCATCTCTATCATGATTATATAG 3′

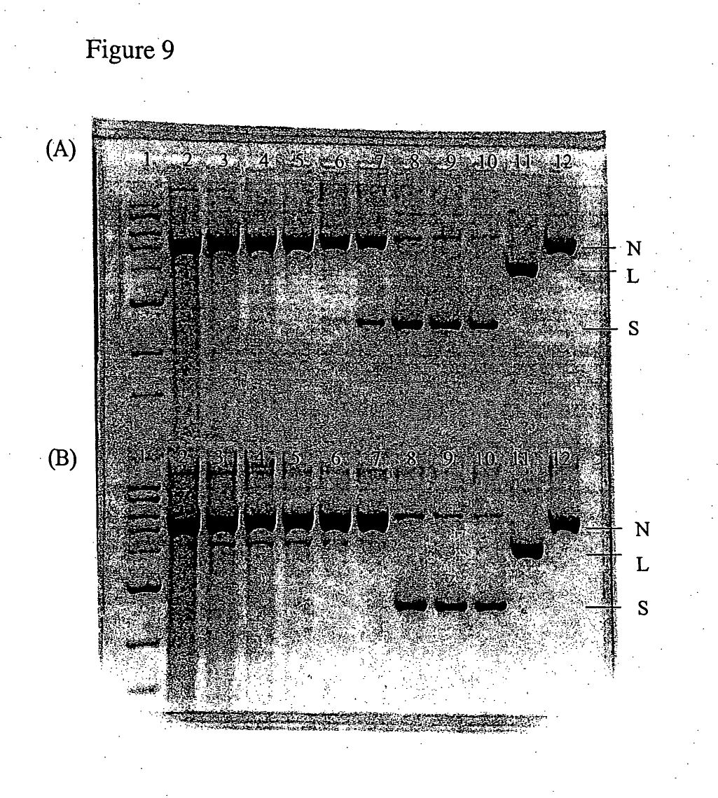

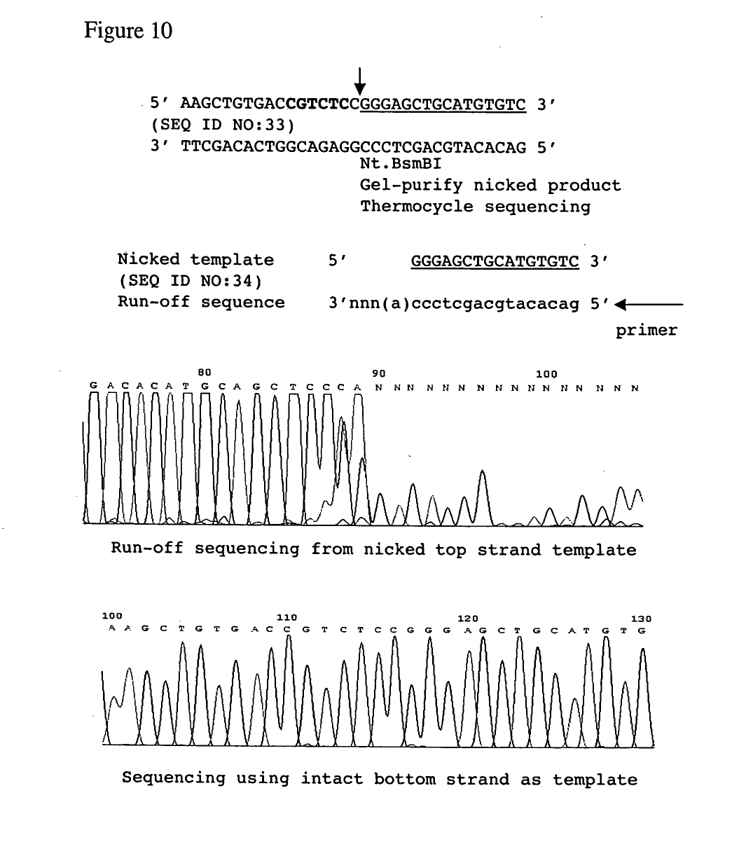

Inverse PCR was carried out under the conditions of 94° C. for 30 sec, 55° C. for 30 sec, 72° C. for 4 min for 20 cycles (Deep Vent DNA polymerase, in the presence of 2 and 4 mM MgSO₄). Amplified PCR product was digested with DpnI and transferred into E. coli expression host ER2683 [pACYC-BsmAIM]. Individual transformants were cultured in 10 ml LB+Ap and Cm and shaken overnight at 37° C. Cell cultures (10 ml) were directly screened for BsmBI nicking enzyme activity. Six clones were screened for N.BsmBI nicking activity and three clones showed nicking activity. After the nicking activity was found the cell pellet was resuspended in a sonication buffer and lysed by sonication. Cell debris was removed by centrifugation and the cell extract was used to nick (digest) supercoiled pBR322 DNA. BsmBI variant R233D displayed DNA nicking activity in both cell culture and cell extract (FIG. 9B). The nicked product was gel-purified and used as the template for run-off sequencing. It was determined that BsmBI R233D nicked the top strand and thus it was named Nt.BsmBI (R233D). The result of nicking site determination is shown in FIG. 10. The bottom strand template produced a continuous primer extension product while the top strand template resulted in a truncated product.

BsaI nicking variants Nb.BsaI (N441D/R442G) and Nb.BsaI (R442G) carried aa substitutions at N441 and R442. In BsaI endonuclease The R442G substitution is the critical residue that converted BsaI endonuclease to N.BsaI nicking enzyme. The corresponding aa residue in BsmBI is Arg438 (A Glu residue precedes Arg 438, Glu437Arg438, FIG. 8). The Arg438 was changed to Asp or Gly by oligonucleotide-directed mutagenesis (inverse PCR). The inverse PCR primers have the following sequences:

(SEQ ID NO:19)

R438G forward primer
5′ AATTCTAAGGATATAAATGAGGGAAAGTTAATTAAATTTGACACT

3′
(SEQ ID NO:20)

R438G reverse primer
5′ AGT GTCAAATTTAATTAACTTTCCCTCATTTATATCCTTAGAATT

3′
(SEQ ID NO:21)

R438D forward primer
5′AATTCTAAGGATATAAATGAGGATAAGTTAATTAAATTTGACACT 3′

(SEQ ID NO:22)

R438D reverse primer
5′ AGTGTCAAATTTAATTAACTTATCCTCATTTATATCCTTAGAATT

3′

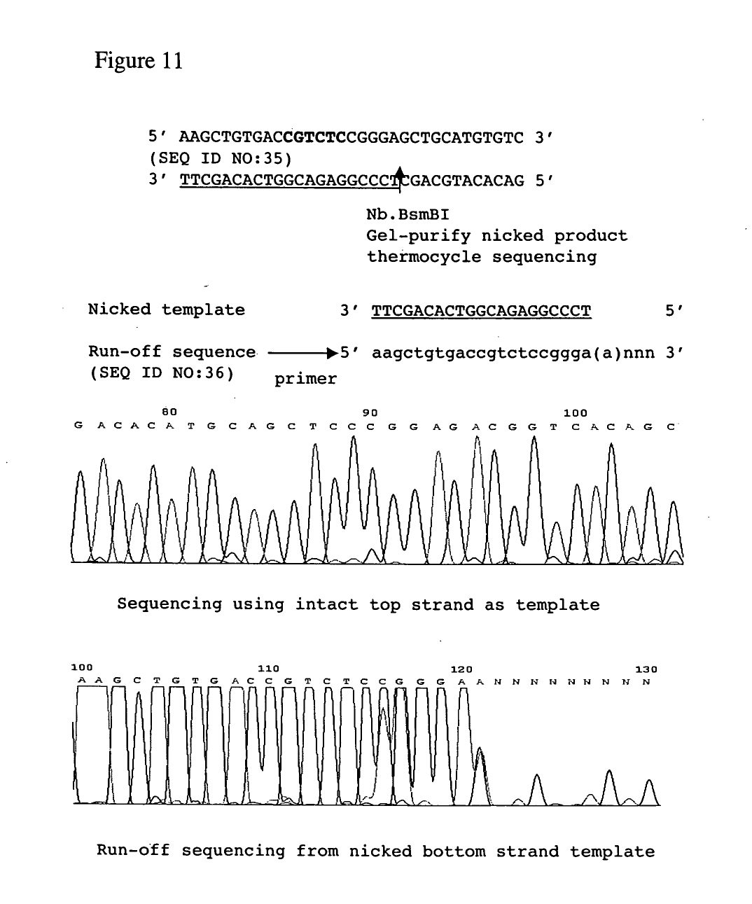

Inverse PCR was carried out under the conditions of 94° C. for 30 sec, 55° C. for 30 sec, 72° C. for 4 min for 20 cycles (Deep Vent DNA polymerase, in the presence of 2 and 4 mM MgSO₄). Amplified PCR product was digested with DpnI and transferred into E. coli expression host ER2683 [pACYC-BsmAIM]. Individual transformants were cultured in 10 ml LB+Ap and Cm and shaken overnight at 37° C. Cell cultures were used to screen BsmBI nicking enzyme activity. After detection of nicking activity the cell cultures were subjected to low speed centrifugation and cell pellets were resuspended in a sonication buffer and lysed by sonication. Cell debris was removed by centrifugation and the cell extract was used to nick (digest) supercoiled pBR322 DNA. BsmBI variants R438D and R438G displayed DNA nicking activity in cell culture and in cell extract (FIG. 9A). The nicked product was gel-purified and used as the template for run-off sequencing. It was determined that BsmBI R438D nicked the bottom strand and thus it was named Nb.BsmBI (R438D). The result of nicking site determination is shown in FIG. 11. The top strand was not nicked and the run-off sequence was read continuously. The bottom strand was nicked and the run-off sequence suddenly stops at the nicked site.

### Example 3

### Engineering Strand-Specific Nicking Enzyme Nt.BsmAI and Nb.BsmAI from BsmAI Restriction Endonuclease by Site-Directed Mutagenesis

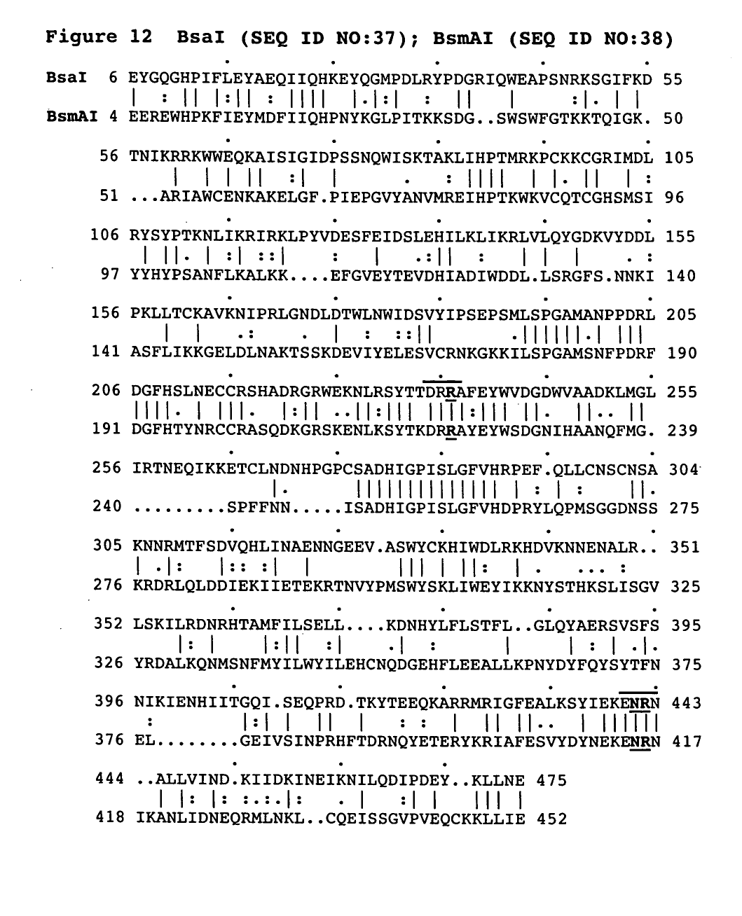

The BsaI and BsmAI restriction endonucleases recognize similar DNA recognition sequence (BsaI GGTCTC; BsmAI GTCTC) and cleave N1/N5 downstream. The two endonucleases show a 48% percentage of aa sequence similarity and 37% aa sequence identity (FIG. 12). It was reasoned that the aa substitutions identified in BsaI that converted BsaI endonuclease to nicking enzymes could be directly introduced in the BsmAI restriction endonuclease by site-directed mutagenesis to generate BsmAI nicking variants. The aa sequence alignment program “Bestfit” of GCG shows that the residue Arg221 of BsmAI corresponds to the Arg236 of BsaI (FIG. 12). As demonstrated in Exampe 1, Arg236 to Gly or Arg236 to Asp in BsaI endonuclease generated BsaI nicking variants (Nb.BsaI R236D, Nb.BsaIR236G). A corresponding BsmAI variant R221D was constructed by oligonucleotide-directed mutagenesis (inverse PCR). The bsmAIR gene was first cloned in pUC19 vector to generate pUC-BsmAIR. The inverse PCR primers have the following sequences:

(SEQ ID NO:23)

R221D forward primer
5′ TCGTATACAAAAGATAGAGATGCATATGAATATTGGAGC 3′

(SEQ ID NO:24)

R221D reverse primer
5′ GCTCCAATATTCATATGCATCTCTATCTTTTGTATACGA 3′

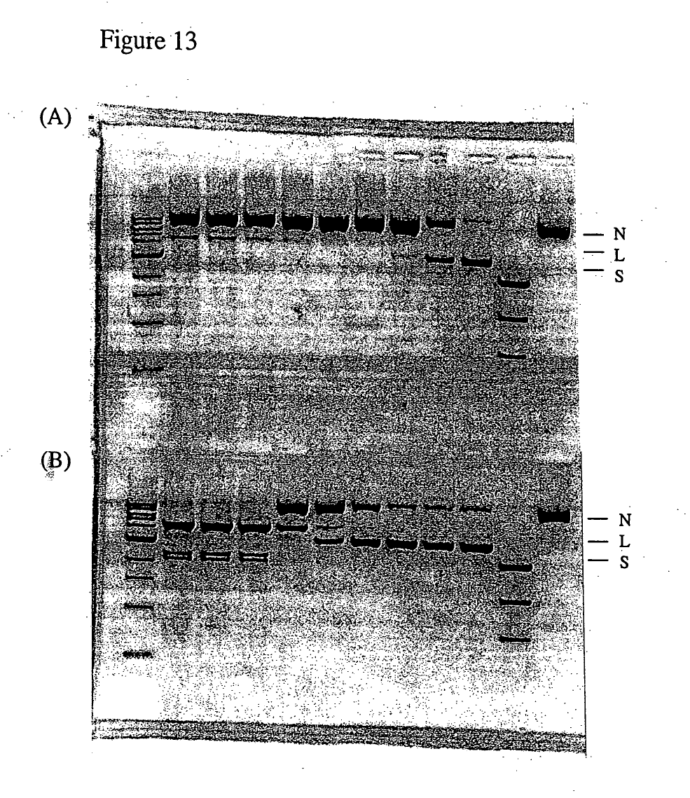

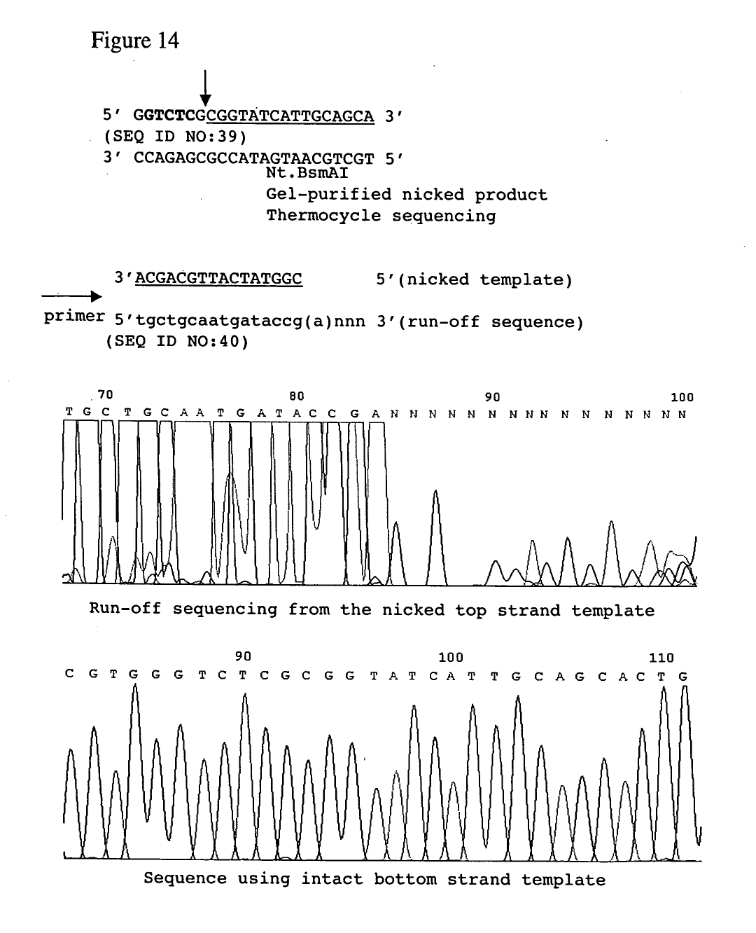

Inverse PCR was carried out under the conditions of 94° C. for 30 sec, 55° C. for 30 sec, 72° C. for 4 min and 7 sec for 20 cycles (Deep Vent DNA polymerase, in the presence of 2, 4, and 6 mM MgSO₄). Amplified PCR product was digested with DpnI and transferred into E. coli expression host ER2683 [pACYC-BsmAIM]. Individual transformants were cultured in 10 ml+Ap and Cm and shaken overnight at 37° C. Cell cultures were screened for BsmAI nicking enzyme activity. Sixteen clones were screened for N.BsmAI nicking activity and 14 clones showed nicking activity. Cell pellets of the positive clones were resuspended in a sonication buffer and lysed by sonication. Cell debris was removed by centrifugation and the cell extract was used to nick (digest) supercoiled pBR322 DNA. BsmAI variant R221D displayed DNA nicking activity in cell culture and in cell extract (FIG. 13A). A small amount of dsDNA cleavage activity was also detected (FIG. 13A). The nicked product was gel-purified and used as the template for run-off sequencing. It was determined that BsmAI (R221D) nicked the top strand and thus it was named Nt.BsmAI (R221D). The result of nicking site determination is shown in FIG. 14.

BsaI nicking variants Nb.BsaI (N441D/R442G) and Nb.BsaI (R442G) carried aa substitutions at N441 and R442. In BsaI endonuclease, the R442G substitution is the critical residue that converted BsaI endonuclease to N.BsaI nicking enzyme. The corresponding aa residues in BsmAI are N415 and R416. Asn415 to Asp (N415D) and Arg416 to Gly (R416G) changes were introduced into BsmAI endonuclease by oligonucleotide-directed mutagenesis (inverse PCR). The inverse PCR primers have the following sequences:

(SEQ ID NO:25)

N415D/R416G forward primer
5′ GATTATAATGAAAAAGAAGATGGAAATATAAAAGCAAACCTC 3′

(SEQ ID NO:26)

N415D/R416G reverse primer
5′ GAGGTTTGCTTTTATATTTCCATCTTCTTTTTCATTATAATC 3′

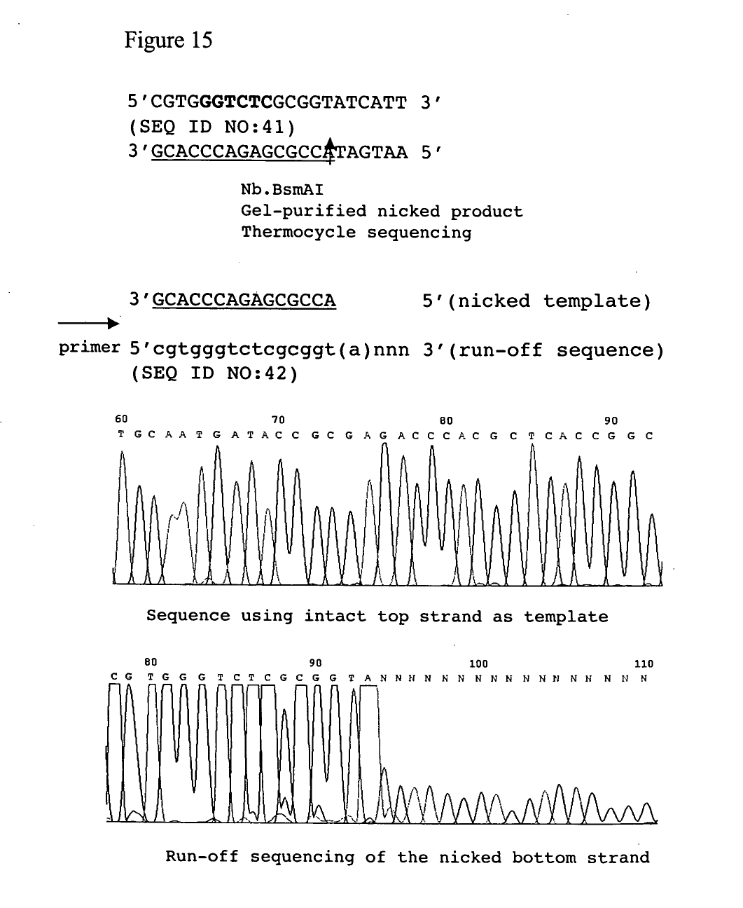

Inverse PCR was carried out under the conditions of 94° C. for 30 sec, 55° C. for 30 sec, 72° C. for 4 min and 7 sec for 20 cycles (Deep Vent DNA polymerase, in the presence of 2, 4, and 6 mM MgSO₄). Amplified PCR product was digested with DpnI and transferred into E. coli expression host ER2683 [pACYC-BsmAIM]. Individual transformants were cultured in 10 ml LB+Ap and Cm and shaken overnight at 37° C. The cell cultures were screened for BsmAI nicking enzyme activity. Six positive nicking activities were found among 8 screened. The cell pellets of the six positive clones were resuspended in a sonication buffer and lysed by sonication. Cell debris was removed by centrifugation and the cell extract was used to nick (digest) supercoiled pBR322 DNA. BsmAI variant N415D/R416G displayed both dsDNA cleavage and DNA nicking activity (FIG. 13B). Since N.BsmAI (N415D/R416G) displayed dsDNA cleavage activity in addition to nicking, the strand-specific nicking activity can be further optimized. The nicking strand of BsmAI variant N415D/R416G was determined by run-off sequencing. It nicks the bottom strand as indicated by the sudden decrease in sequence peak signal at the nicked site (FIG. 15). The top strand generated normal DNA sequence.

### Example 4

### Engineering Strand-Specific Nicking Enzyme N.BsmAI from BsmAI Restriction Endonuclease by Random Mutagenesis and Genetic Screen

Random mutagenesis of the bsmAIR gene was performed to isolate more BsmAI nicking variants. The bsmAIR gene was mutagenized by error-prone inverse PCR under the condition of 94° C. for 30 sec, 55° C. for 30 sec, 72° C. for 3 min 54 sec for 25 cycles with Taq DNA polymerase in the presence of 2, 4, 6, 10 mM MgCl₂, dNTP, pUC-BsmAI template, and 1× Thermopol buffer. PCR products were pooled and purified by Qiagen spin columns and digested with XhoI and DpnI. DpnI digestion eliminated the Dam-methylated template DNA. The PCR product was self-ligated and transferred into E. coli expression host ER2566 by electroporation and transformants were plated on Ap plates. The recipient host did not carry a protection methylase and therefore wt enzyme or mutant enzymes with high activities killed the host cells. Only null mutants, mutants with low activity, or mutants with cleavage deficiency and binding proficiency survived this transformation step. A total of approximately 3,600 survivor transformants were obtained and pooled together. Plasmid DNA was prepared from pooled cells to form a mutant plasmid DNA library.

The BsmAI wt and the mutant plasmids (˜3.9 kb) were digested with restriction enzymes PstI and BseYI. PstI was located near the middle of the bsmAIR gene and PstI digestion split the endonuclease gene into two segments (43% and 57%). BseYI is located in the ColEI replication origin of the vector. The restriction fragments were gel-purified from a low-melting agarose gel and further purified by Qiagen spin columns. The wt N-terminal coding sequence (wt left half) was combined with the mutagenized (inactivated) C-terminal coding sequence (mutant right half) and ligated with T4 DNA ligase. Conversely, the mutagnized (inactivated) N-terminal coding sequence (mutant left half) was combined with the wt C-terminal coding sequence (wt right half) and ligated with T4 DNA ligase. The ligated DNA was transferred into E. coli expression host ER2683 [pACYC-BsmAI] by electroporation. The cognate methylase M.BsmAI conferred host DNA protection. Transformants were plated on LB agar plates+Ap and Cm and incubated overnight at 37° C.

Single colonies of transformants were inoculated into 10 ml of LB+Ap and Cm and cultured overnight in a 37° C. shaker. Ten ml of total cell culture (cells plus media) were used to nick (digest) 1 mg of pBR322 supercoiled DNA in a nicking assay. The nicked (digested) DNA was analyzed by agarose gel electrophoresis. When nicked circular DNA was detected in the cell culture of a particular isolate, 10 ml of cells were pelleted by low speed centrifugation. The cell pellet was resuspended in a sonication buffer and lysed by sonication. The clarified supernatant (cell extract) was used to confirm the nicking enzyme activity. A total of 240 transformants were screened for nicking activity. Three clones (#48, #100, and #234) displayed nicking activity in cell cell culture and in cell extracts. The plasmid DNA was purified from these three isolates and the bsmAIR allele was sequenced with pUC universal primers and custom-made primers: The primers have the following sequences:

(SEQ ID NO:27)
5′ GAAGTTATTTATGAACTTGAATCA 3′ (303-023)
(SEQ ID NO:28)
5′ TTAATTTCTGGAGTTTACCGAGAT 3′ (303-024)

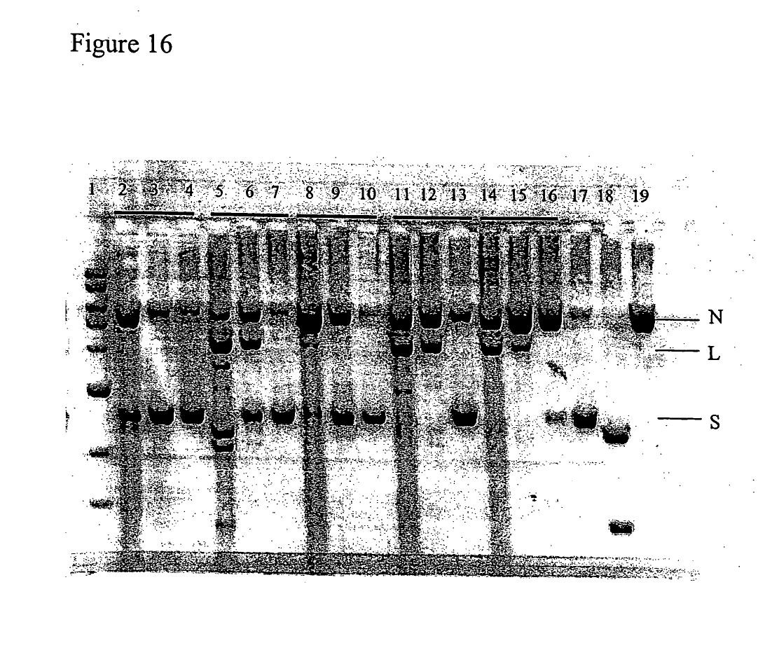

BsmAI nicking variant #48 carries a 59-aa C-terminal deletion as the result of introducing a stop codon at codon 394 [#48 N.BsmAI D(394-465)] (see FIG. 16, lanes 5-7). Another BsmAI nicking variant #100 carries two aa substitutions Nt.BsmAI (R221H/K287N). As demonstrated in Example 3, N.BsmAI variant R221D is a top strand nicking enzyme (Nt.BsmAI). Consistent with result presented in Example 3, the random mutagenesis method also identified R221 as the critical residue for conversion to a nicking enzyme. The contribution of K287N substitution to nicking activity is probably minimal in the double mutant.

Since the first genetic screen only identified three BsmAI nicking variants (#48, #100, and #234) and these three enzymes also displayed some dsDNA cleavage activity, a second genetic screen was carried out to isolate more nicking variants. The BsmAI wt and the mutant plasmids (˜3.9 kb) were digested with restriction enzymes BsrGI and BseYI. BsrGI was located near the middle of the bsmAIR gene and BsrGI digestion split the endonuclease gene into two segments (56% and ⁴⁴%). BseYI is located in the ColE1 replication origin of the vector. The restriction fragments were gel-purified from a low-melting agarose gel and further purified by Qiagen spin columns. The wt N-terminal coding sequence (wt left half) was combined with the mutagenized (inactivated) C-terminal coding sequence (mutant right half) and ligated with T4 DNA ligase. Conversely, the mutagnized (inactivated) N-terminal coding sequence (mutant left half) was combined with the wt C-terminal coding sequence (wt right half) and ligated with T4 DNA ligase. The ligated DNA was transferred into E. Coli expression host ER2683 [pACYC-BsmAI] by electroporation. The cognate methylase M.BsmAI conferred host DNA protection. Transformants were plated on LB agar plates+Ap and Cm and incubated overnight at 37° C. A total of 240 cell cultures of transformants were screened for DNA nicking activity on supercoiled pBR322 DNA. Sixteen samples showed DNA nicking activities. Cell extracts with DNA nicking activity were further confirmed and the four most active variants (#40, #55, # 101, and #197) were selected for DNA sequencing. The DNA nicking activity of the N.BsmAI variants from genetic screen 2 were shown in FIG. 16 (#40, lanes 2-4; #55, lanes 8-10; #101, lanes 11-13; #197, lanes 14-16). The aa substitutions or deletion in the N.BsmAI nicking variants are shown below in Table 2:

TABLE 2
Amino acid substitutions or deletion in N.BsmAI nicking variants

| | | Aa | |
|---|---|---|---|
| Isolate number | Codon change | substitution/del | Nicking strand |
| #48 (screen 1) | UAU to UAA | D(394-465) | Not |
| | | | determined |
| #100 (screen 1) | CGU to CAU | R221H/K287N | Top |
| | AAA to AAC | | |
| #40 (screen 2) | AAC to UAC | N349Y | Top |
| #55 (screen 2) | UGG to UGU | W8C/G207E | Top |
| | GGA to GAA | | |
| #101 (screen 2) | AAG to UAG | D(362-465) | Top |
| #197 (screen 2) | CCC to CAC | P267H/W302L/R3 | Top |
| | UGG to UUG | 86S | |
| | CGU to AGU | | |

The deletion variant #101 D(362-465) and mutant #197 displayed nicking activity when limited digestion was performed (at 1/100 dilution in FIG. 16). In high enzyme concentration, there exists dsDNA cleavage activity. Therefore, the nicking activity can be further optimized by mutagenesis of Arg386, for example, isolation of R386D or R386E. In addition, the aa substitutions identified by random mutagenesis may be combined to generate double or triple mutants, for example, isolation of N349Y/R386S, G207E/R386S, or G207E/N349Y/R386S. The double or triple mutants can be constructed by oligonucleotide-directed PCR mutagenesis.

### Example 5

### Localized Random Mutagenesis of Type IIA/IIS Restriction Endonuclease to Isolate Strand-Specific Nicking Enzymes

A Type IIA/IIS restriction endonuclease gene can be digested by restriction enzymes to split into two fragments, one fragment coding for the N-terminal aa and the other half encoding the C-terminal aa. The internal restriction site could be located in 5% to 95% of the coding sequence in relative position to the first start codon. The N-terminal coding DNA fragment or the C-terminal fragment can be cloned into an expression vector. The N-terminal coding DNA can be mutagenized by random mutagenesis methods. After restriction digestion to generate an appropriate fragment, the mutagenized fragment can be ligated to the wt C-terminal coding fragment/vector and the ligated DNA used to transform a pre-modified or unmodified host. Nicking enzyme variants can be screened from the cell cultures or cell extracts of the transformants. Alternatively, the C-terminal coding DNA can be mutagenized by random mutagenesis method. After restriction digestion to generate appropriate flanking restriction sites, the mutagenized fragment can be ligated to the wt N-terminal coding fragment/vector and the ligated DNA is used to transform a pre-modified host. Nicking enzyme variants can be screened from the cell cultures or cell extracts of the transformants. After the nicking variants are isolated the nicking site and nicking strand can be determined on appropriate DNA substrates.

### Example 6

### Random Mutagenesis of a Portion of Type IIA/IIS Restriction Endonuclease Gene to Isolate Strand-Specific Nicking Enzymes

One can clone the wt Type IIA/IIS restriction endonuclease gene into a plasmid and replicate the plasmid in a host cell with a protective methylase. One can clone a portion of restriction endonuclease gene (a restriction fragment that covers 5% to 95% of the entire gene) and then mutagenize the sub-fragment by error-prone PCR or any other random mutagenesis methods. After restriction digestion to generate appropriate restriction ends, the mutagenized fragment is then ligated to the wt DNA/vector, replacing the corresponding wt region. The ligated DNA is used to transform a host with or without a protective methylase. Nicking enzyme variants can be screened from the cell cultures of the transformants.

1. A method for engineering a strand-specific nicking endonuclease, comprising:
(a) transforming a first host cell population lacking methylase protection, with plasmids containing a randomly mutagenized restriction endonuclease gene;
(b) culturing the transformed host cells of step (a) and isolating the plasmids therefrom;
(c) cleaving the mutagenized restriction endonuclease gene of step (b) and a corresponding wild-type restriction endonuclease gene into fragments;
(d) performing an in vitro backcross between the wild-type and mutagenized restriction endonuclease fragments of step (c) and obtaining a ligated gene;
(e) detecting a strand-specific nicking activity of a protein expressed by the ligated gene of step (d); and
(f) identifying the engineered strand-specific nicking endonuclease.

2. A method according to claim 1, wherein cleaving the restriction endonuclease gene of step (c) occurs by means of restriction endonuclease digestion and wherein the restriction endonuclease gene fragments are purified on an agarose gel.

3. A method according to claim 1, wherein step (d) further comprises: transforming a second population of host cells with the ligated gene wherein the transformants are protected by cognate or non-cognate methylases.

4. A method according to claim 3, further comprising, forming colonies from individual transformants.

5. A method according to claim 4, wherein the colonies are individually screened for nicking activity using a supercoiled DNA substrate.

6. A method according to claim 5, wherein the step of individual screening utilizes total cells in a culture media or a cell extract.

7. A method according to claim 1, wherein step (f) further comprises determining the position and type of mutation in the DNA encoding the nicking endonuclease.

8. A method according to claim 1, wherein the mutagenized restriction endonuclease gene has a deletion, an insertion or a substitution of one or more nucleotides.

9. A method according to claim 1, wherein the mutagenized gene have a plurality of mutations.

10. A method according to claim 1, wherein mutagenized gene has a single mutation.

11. A method according to claim 1, wherein the restriction endonuclease gene encodes a protein having a C-terminal end and an N-terminal end such that one or more mutations are located at the C-terminal end.

12. A method according to claim 1, the mutagenized gene having a deletion in the range of 3 to 600 nucleotides.

13. A method according to claim 1, wherein the host cell preparation of step (a) is selected from a gram negative or a gram positive bacterial host.

14. A method according to claim 1, wherein the host cell preparation of step (a) is selected from E. coli or a Bacillus strain.

15. A method according to claim 1, further comprising:
identifying the mutation in the nicking endonuclease compared with the wild-type restriction endonuclease from which it is derived and introducing the mutation by site-directed mutagenesis into an isochizomer or neoschizomer of the restriction endonuclease.

16. A method according to claim 1, further comprising introducing an additional mutation into the nicking endonuclease of step (f) by site-directed mutagenesis for enhancing nicking activity or minimizing double strand DNA cleavage activity or both.

17. A method according to claim 1, wherein the mutagenized restriction endonuclease gene is a Type IIA endonuclease gene.

18. A method according to claim 1, wherein the nicking endonuclease is a thermophilic nicking endonuclease.

19. A method according to claim 17, wherein the restriction endonuclease is BsaI, BsmAI BsmBI, or neoschizomers or isoschizomers thereof.

20. A method according to claim 1, wherein step (f) further comprises determining the duplex DNA strand specificity of the nicking endonuclease.

21. A method according to claim 1, wherein the nicking endonuclease of step (d) is a top strand nicking endonuclease.

22. A method according to claim 1, wherein the nicking endonuclease of step (d) is a bottom strand nicking endonuclease.

23. A nicking endonuclease made according to claim 1.

24. A nicking endonuclease comprising a modified recombinant BsaI.

25. A nicking endonuclease comprising a modified recombinant BsmAI.

26. A nicking endonuclease comprising a modified recombinant BsmBI.

27. A method for introducing one or more site-specific nicks into pre-selected strands of a DNA duplex, the method comprising: digesting the DNA duplex with a nicking endonuclease made according to claim 19 under conditions permitting nicking activity.

28. A method for amplifying a target sequence comprising:
(a) providing a single-stranded nucleic acid fragment containing the target sequence, the fragment having a 5′ end and a 3′ end;
(b) binding an amplification primer for SDA to the 3′ end of the fragment such that the primer forms a 5′ single-stranded overhang, the amplification primer comprising a recognition/cleavage site for a synthetic nicking endonuclease made according to claim 1, and;
(c) extending the amplification primer on the fragment in the absence of a derivatized or substituted deoxynucleoside triphosphate and in the presence of:
(i) a DNA polymerase having strand-displacing activity and lacking 5′-3′ exonuclease activity; and
(ii) four deoxynucleoside triphosphates; and
(d) nicking the amplified double-stranded target sequence with the nicking endonuclease extending from the nick using the DNA polymerase, thereby displacing the first newly synthesized strand from the fragment and generating a second extension product comprising a second newly synthesized strand; and repeating the nicking, extending and displacing steps such that the target sequence is amplified.

29. A method for engineering an enzyme with at least one of a modified substrate specificity and activity, comprising:
(a) forming a randomly mutagenized DNA library wherein the library has one or more genes encoding whole or part of a mutant enzyme, the mutant enzyme being substantially inactive, the substantially inactive enzyme having an N-terminal end and a C-terminal end, wherein the inactivation results from a mutation in the N-terminal end or C-terminal end of a wild-type enzyme;
(b) cleaving the one or more genes expressing the inactive endonuclease into at least a first fragment and a second fragment, wherein the first fragment encodes the C-terminal end of the enzyme and the second fragment encodes the N-terminal end of the enzyme;
(c) performing a ligation between fragments selected from: the first fragment and a third fragment encoding an N-terminal end of the wild-type enzyme; the second fragment with a fourth fragment encoding the C-terminal end of the wild-type enzyme; or both first and second fragments to third and fourth fragments respectively; and
(d) expressing the ligated DNA in a host cell to obtain the enzyme having modified substrate specificity and activity.

30. A method of amplifying a target nucleic acid, comprising:
(a) nicking at least one strand of a double-stranded target nucleic acid at a plurality of sites with a nicking enzyme made according to claim 1 to form at least two new 3′ termini;
(b) extending one or more of the at least two new 3′ termini with a DNA polymerase;
(c) nicking the extension product of step (b); and
(d) extending the nicking product of step (c) to amplify at least a portion of one strand of the target nucleic acid.

31. A method of rapidly screening nicking enzyme variants using host cells plus culturing media in a DNA nicking reaction containing a supercoiled DNA substrate.
